(19) United States
(12) Patent Application Publication
(10) Pub. No.: US 2023/0255768 A1
(43) Pub. Date: Aug. 17, 2023

(54) EXPANDABLE SHEATH FOR INTRODUCING AN ENDOVASCULAR DELIVERY DEVICE INTO A BODY

(71) Applicant: EDWARDS LIFESCIENCES CORPORATION, Irvine, CA (US)

(72) Inventors: Ilan Tamir, Irvine, CA (US); David A. Rezac, Lancaster, MA (US); Devin H. Marr, Westford, MA (US); Jack Sattell, Boston, MA (US); Michelle Riches, Leominster, MA (US); Bridget Bergstrom, Maple Grove, MN (US); Matthew Lapointe, Natick, MA (US)

(21) Appl. No.: 18/138,943

(22) Filed: Apr. 25, 2023

Related U.S. Application Data
(63) Continuation of application No. PCT/US2021/058060, filed on Nov. 4, 2021.
(60) Provisional application No. 63/109,800, filed on Nov. 4, 2020.

(51) Int. Cl. A61F 2/24; A61M 25/00

(52) U.S. Cl. CPC A61F 2/2436; A61M 25/0023; A61M 2025/0024

(57) ABSTRACT

Disclosed herein is an expandable sheath that can be used in conjunction with a catheter assembly to introduce a prosthetic device, such as a heart valve, into a patient. The disclosed herein sheath can minimize trauma to the vessel by allowing for temporary expansion of a portion of the introducer sheath to accommodate the delivery apparatus, followed by a return to the original diameter once the prosthetic device passes through. Some aspects can include a sheath comprising an inner layer comprising a reinforcing layer comprising a plurality of struts that can be encapsulated within a polymer layer comprising a tether portion and an outer layer disposed over the inner layer. The disclosed sheath is configured to locally expand from a predetermined first diameter d₁ to an expanded second diameter d₂ during application of a radial outward force by passage of a medical device through the sheath.

### CROSS-REFERENCE TO RELATED APPLICATIONS

This application is a continuation of International Application No. PCT/US2021/058060, filed Nov. 4, 2021, which claims the benefit of U.S. Provisional Application No. 63/109,800, filed Nov. 4, 2020, the contents of which are incorporated herein by reference in their entirety.

### FIELD

The present application concerns aspects of a sheath for use with catheter-based technologies for repairing and/or replacing heart valves and delivering a prosthetic device, such as a prosthetic valve to a heart via the patient's vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

An introducer sheath can be used to safely introduce a delivery apparatus into a patient's vasculature (e.g., the femoral artery). An introducer sheath generally has an elongated sleeve that is inserted into the vasculature and a housing that contains one or more sealing valves that allow a delivery apparatus to be placed in fluid communication with the vasculature with minimal blood loss. A conventional introducer sheath typically requires a tubular loader to be inserted through the seals in the housing to provide an unobstructed path through the housing for a valve mounted on a balloon catheter. A conventional loader extends from the proximal end of the introducer sheath, and therefore decreases the available working length of the delivery apparatus that can be inserted through the sheath and into the body.

Conventional methods of accessing a vessel, such as a femoral artery, prior to introducing the delivery system include dilating the vessel using multiple dilators or sheaths that progressively increase in diameter. This repeated insertion and vessel dilation can increase the amount of time the procedure takes and the risk of damage to the vessel.

Radially expanding intravascular sheaths have been disclosed. Such sheaths tend to have complex mechanisms, such as ratcheting mechanisms that maintain the shaft or sheath in an expanded configuration once a device with a larger diameter than the sheath's original diameter is introduced.

However, delivery and/or removal of prosthetic devices and other material to or from a patient still poses a significant risk to the patient. Furthermore, accessing the vessel remains a challenge due to the relatively large profile of the delivery system that can cause longitudinal and radial tearing of the vessel during insertion. The delivery system can additionally dislodge calcified plaque within the vessels, posing an additional risk of clots caused by the dislodged plaque.

Accordingly, there remains a need in the art for an improved introducer sheath for endovascular systems used for implanting valves and other prosthetic devices.

### SUMMARY

Aspects of the present disclosure are directed to an expandable sheath that can minimize trauma to the vessel by allowing for temporary expansion of a portion of the introducer sheath to accommodate a delivery system, followed by a return to the original diameter once the delivery system passes through. Some aspects can comprise a sheath with a smaller profile than that of prior art introducer sheaths. Furthermore, certain aspects described herein can reduce the length of time a procedure takes and reduce the risk of a longitudinal or radial vessel tear or plaque dislodgement because only one sheath is required, rather than several different sizes of sheaths. The disclosed aspects can require only a single vessel insertion, as opposed to requiring multiple insertions for the dilation of the vessel.

In some aspects, disclosed is a sheath for delivering a medical device, wherein the sheath has a proximal and a distal end; wherein the sheath has a collapsed unexpanded state and is configured to expand to an expanded state under passage of a medical device and resiliently return to the collapsed unexpanded state after passage of the medical device, wherein the sheath comprises: a) an inner layer comprising: i) a reinforcing layer comprising a plurality of struts axially extending from the proximal end to the distal end and circumferentially arranged in a predetermined pattern; ii) a polymer layer disposed around the plurality of struts to form a plurality of encapsulated struts; and wherein the polymer layer further comprises a plurality of tether portions circumferentially connecting each of the plurality of struts to each other; wherein when the sheath is in the unexpanded state the plurality of encapsulated struts are configured to collapse to a randomized or an aligned organization to form a sheath defining a cross-section having a first diameter; wherein the plurality of tether portions at least partially folded between two adjacent struts of the plurality of encapsulated struts; and wherein the sheath is in the expanded state, the plurality of encapsulated struts and the plurality of tether portions together configured to organize to form a sheath having a substantially circular cross-section having a second diameter; wherein the second dimeter is larger than the first diameter; and b) an outer layer disposed over the inner layer of the sheath and configured to maintain a general shape of the sheath; wherein the sheath is configured to resist axial elongational of the sheath such that a length of the sheath remains substantially constant.

In some aspects, at least a portion of the plurality of struts can comprise struts that are not coupled to any adjacent struts other than through the circumferentially connecting tethering portions of the polymer layer encapsulating the plurality of struts.

In certain aspects, the reinforcing layer can comprise a plurality of individual wires forming the plurality of struts. Yet, in other aspects, the reinforcing layer can be an etched sheet forming the plurality of struts. While in still further exemplary aspects, the reinforcing layer can be a laser-cut sheet forming the plurality of struts. While in still further exemplary aspects, the reinforcing layer can be a laser-cut tube forming the plurality of struts. In one exemplary aspect, the tube is hypotube.

In still further aspects, the sheath disclosed herein is configured to receive an introducer configured to reinforce a radial orientation of the plurality of encapsulated struts upon introducing the sheath into a body. Yet, in other exemplary aspects, if the introducer is present, the introducer can comprise at least a partially grooved surface configured to match the radial orientation of the plurality of encapsulated struts.

Also disclosed herein are aspects comprising methods of making a sheath for delivering a medical device. In certain aspects, the method of making such a sheath comprises a) providing an inner layer comprising: i) a reinforcing layer comprising a plurality of struts axially extending from the proximal end to the distal end and circumferentially arranged in a predetermined pattern; and ii) a polymer layer disposed around the plurality of struts to form a plurality of encapsulated struts; and wherein the polymer layer further comprises a plurality of tether portions circumferentially connecting each of the plurality of struts to each other; b) disposing an outer layer around the inner layer such that the outer layer is configured to maintain a general shape of the sheath; such that when the formed sheath is in the unexpanded state the plurality of encapsulated struts are configured to collapse to a randomized or an aligned organization to form a sheath defining a cross-section having a first diameter; wherein the plurality of tether portions at least partially folded between two adjacent struts of the plurality of encapsulated struts; and when the sheath is in the expanded state, the plurality of encapsulated struts and the plurality of tether portions together configured to organize to form a sheath having a substantially circular cross-section having a second diameter; wherein the second dimeter is larger than the first diameter; and wherein the formed sheath is configured to resist axial elongational of the sheath such that a length of the sheath remains substantially constant.

Also in some aspects disclosed herein is a method of delivering a medical device through a sheath, the method comprising: a) introducing the medical device into a proximal end of a collapsed sheath, wherein the sheath comprises an inner layer and an outer layer; wherein the inner layer comprises: i) a reinforcing layer comprising a plurality of struts axially extending from the proximal end to the distal end and circumferentially arranged in a predetermined pattern; and ii) a polymer layer disposed around the plurality of struts to form a plurality of encapsulated struts; and wherein the polymer layer further comprises a plurality of tether portions circumferentially; wherein in the collapsed sheath, the plurality of encapsulated struts are in a randomized or an aligned organization forming a sheath defining a cross-section having a first diameter, and wherein the plurality of tether portions at least partially folded between two adjacent struts of the plurality of encapsulated struts; b) advancing the medical device through the sheath such that the medical device exerts a radially outward force on the inner layer, thereby organizing and radially orienting the plurality of encapsulated struts and the plurality of tether portions together forming a sheath having a substantially circular cross-section having a second diameter; wherein the second dimeter is larger than the first diameter; and c) locally contracting the expanded sheath back to an unexpanded configuration by radially compressing the expanded portion with a radially inward bias of an outer layer that extends around the inner layer.

In still further aspects, in the methods disclosed herein, prior to introducing the medical device, an introducer is inserted into the sheath to organize the plurality of encapsulated struts and the plurality of tether portions to form the substantially circular cross-section having a second diameter.

In yet further aspects, the medical device is a prosthetic heart valve mounted in a radially crimped state on a delivery apparatus, and the act of advancing the medical device through the sheath comprises advancing the delivery apparatus and the prosthetic heart valve into the vasculature of a patient.

In some methods, a soft tip portion can be coupled to the distal end of the expandable sheath to facilitate passing the expandable sheath through a patient's vasculature.

The foregoing and other features and advantages of the invention will become more apparent from the following detailed description, which proceeds with reference to the accompanying figures.

### BRIEF DESCRIPTION OF DRAWINGS

FIG. 1 is an elevation view of a sheath according to the present disclosure along with an endovascular delivery apparatus for implanting a prosthetic valve.

FIGS. 2A-2H depict perspective schematics and a cross-section view of an unexpanded sheath in some aspects.

FIG. 3A shows a perspective schematics of an expanded sheath in one aspect. FIG. 3B shows a cross-section view of the expanded sheath depicted in FIG. 3A.

FIGS. 4A-4B depict various exemplary inner layers of exemplary sheaths.

FIGS. 5A-5C depict various exemplary patterns of a reinforcing layer in one aspect.

FIG. 6 shows a photo of a locally expanded exemplary sheath.

FIGS. 7A-7C depict various portions of an exemplary sheath in various aspects.

FIG. 8 depicts an exemplary delivery device.

FIG. 9 depicts a portion of an exemplary sheath along line 36 of FIG. 10.

FIG. 10 depicts a block diagram of one aspect of making a sheath according to the present disclosure.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the present disclosure is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

Unless defined otherwise, all technical and scientific terms used herein have the same meaning as commonly understood by one of ordinary skill in the art to which this invention belongs.

As used in this application and in the claims, the singular forms “a,” “an,” and “the” include the plural forms unless the context clearly dictates otherwise. Thus, for example, reference to a “polymer” includes aspects having two or more such polymers unless the context clearly indicates otherwise.

It is also to be understood that the terminology used herein is for the purpose of describing particular aspects only and is not intended to be limiting. As used in the specification and in the claims, the term “comprising” can include the aspects “consisting of” and “consisting essentially of.” Additionally, the term “includes” means “comprises.”

For the terms “for example” and “such as,” and grammatical equivalences thereof, the phrase “and without limitation” is understood to follow unless explicitly stated otherwise.

References in the specification and concluding claims to parts by weight of a particular element or component in a composition or article, denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

A weight percent of a component, unless specifically stated to the contrary, is based on the total weight of the formulation or composition in which the component is included.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combinations of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the term “substantially,” when used in reference to a composition, refers to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature or component.

As used herein, the term “substantially,” when used in the context of a composition or component of a composition that is substantially absent, is intended to indicate that the recited component is not intentionally batched and added to the composition but can be present as an impurity along with other components being added to the composition. In such aspects, the term “substantially free,” is intended to refer to trace amounts that can be present in the batched components, for example, it can be present in an amount that is less than about 1% by weight, e.g., less than about 0.5% by weight, less than about 0.1% by weight, less than about 0.05% by weight, or less than about 0.01% by weight of the stated material, based on the total weight of the composition.

As used herein, the terms “substantially identical reference composition” or “substantially identical reference article” refer to a reference composition or article comprising substantially identical components in the absence of an inventive component. In another exemplary aspect, the term “substantially,” in, for example, the context “substantially identical reference composition,” refers to a reference composition comprising substantially identical components and wherein an inventive component is substituted with a common in the art component.

Further, the terms “coupled” and “associated” generally mean electrically, electromagnetically, and/or physically (e.g., mechanically or chemically) coupled or linked and does not exclude the presence of intermediate elements between the coupled or associated items.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

As used herein, the term “and/or” includes any and all combinations of one or more of the associated listed items.

It will be understood that, although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example aspects.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein interpreted accordingly.

As used herein, the term “atraumatic” is commonly known in the art and refers to a device or a procedure that minimized tissue injury.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

Although the operations of exemplary aspects of the disclosed method may be described in a particular sequential order for convenient presentation, it should be understood that disclosed aspects can encompass an order of operations other than the particular sequential order disclosed. For example, operations described sequentially may, in some cases, be rearranged or performed concurrently. Further, descriptions and disclosures provided in association with one particular aspect are not limited to that aspect and may be applied to any aspect disclosed.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

Sheath

As disclosed herein, some aspects, an expandable sheath as described herein, can minimize trauma to the vessel by allowing for temporary expansion of a portion of the introducer sheath to accommodate the delivery system, followed by a return to the original diameter once the device passes through. In some aspects, disclosed is a sheath with a smaller profile (e.g., a smaller diameter in the rest configuration) than that of prior art introducer sheaths. Furthermore, present aspects can reduce the length of time a procedure takes, as well as reduce the risk of a longitudinal or radial vessel tear or plaque dislodgement because only one sheath is required, rather than several different sizes of sheaths. Aspects of the present expandable sheath can avoid the need for multiple insertions for the dilation of the vessel. Such expandable sheaths can be useful for many types of minimally invasive surgery, such as any surgery requiring introduction of an apparatus into a subject's vessel. For example, the sheath can be used to introduce other types of delivery apparatus for placing various types of intraluminal devices (e.g., stents, prosthetic heart valves, stented grafts, etc.) into many types of vascular and non-vascular body lumens (e.g., veins, arteries, esophagus, ducts of the biliary tree, intestine, urethra, fallopian tube, other endocrine or exocrine ducts, etc.).

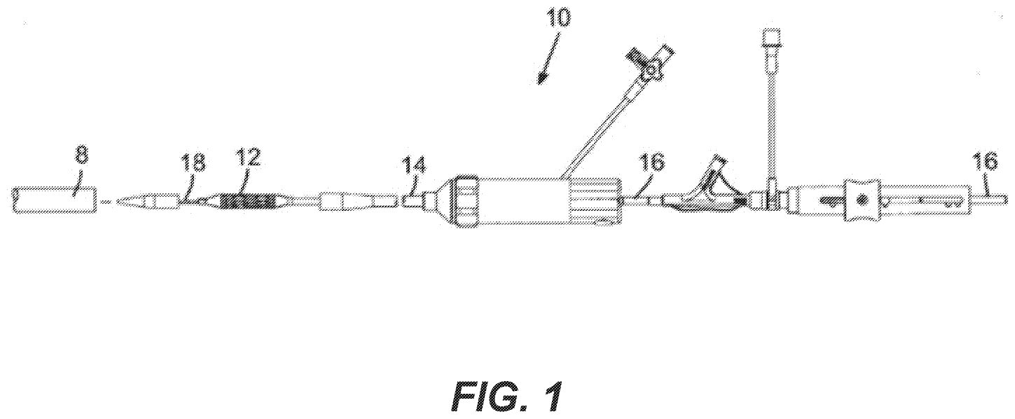

FIG. 1 illustrates a sheath 8 according to the present disclosure, in use with a representative delivery apparatus 10, for delivering a prosthetic device 12, such as a tissue heart valve to a patient. The apparatus 10 can include a steerable guide catheter 14 (also referred to as a flex catheter), a balloon catheter 16 extending through the guide catheter 14, and a nose catheter 18 extending through the balloon catheter 16. The guide catheter 14, the balloon catheter 16, and the nose catheter 18 in the illustrated aspect are adapted to slide longitudinally relative to each other to facilitate delivery and positioning of the valve 12 at an implantation site in a patient's body, as described in detail below. Generally, sheath 8 is inserted into a vessel, such as the transfemoral vessel, passing through the skin of the patient, such that the distal end of the sheath 8 is inserted into the vessel. Sheath 8 can include a hemostasis valve at the opposite, proximal end of the sheath. The delivery apparatus 10 can be inserted into the sheath 8, and the prosthetic device 12 can then be delivered and implanted within the patient.

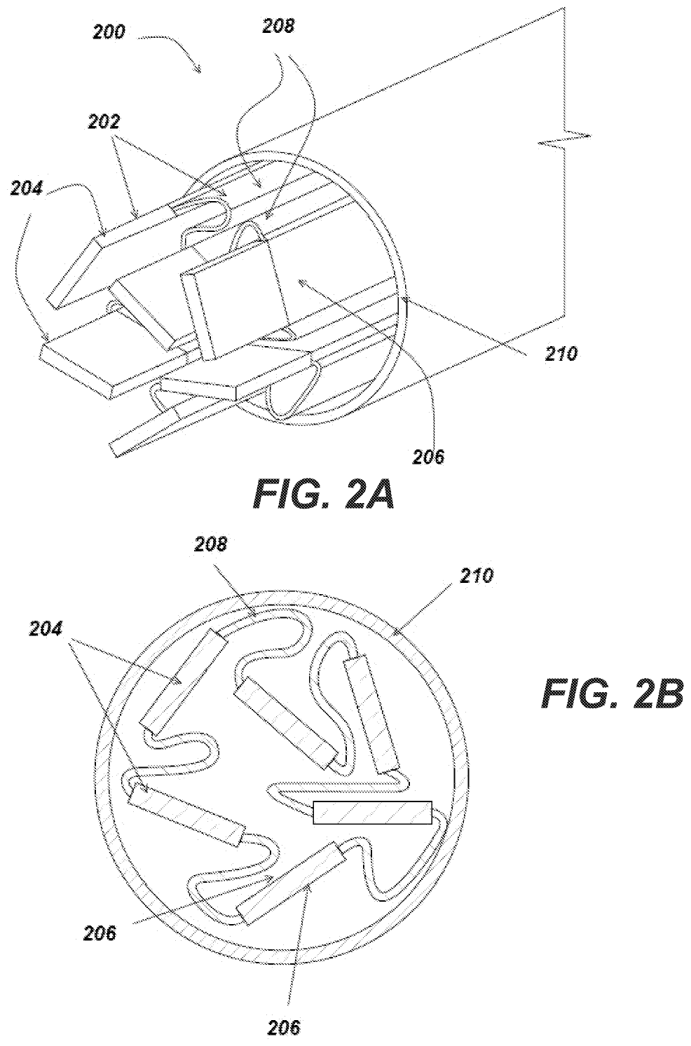

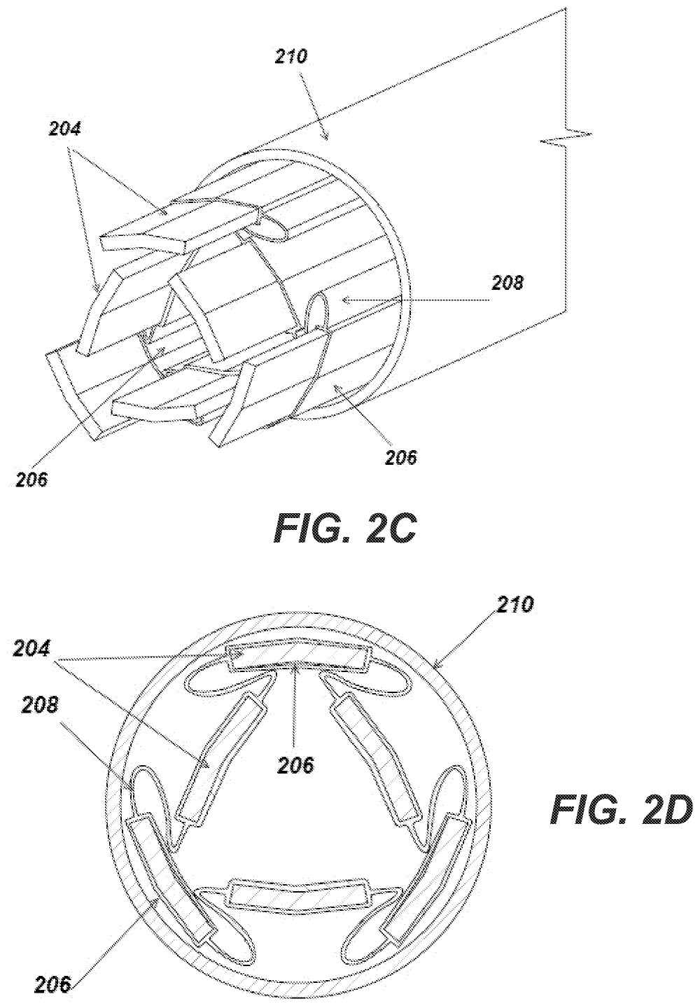

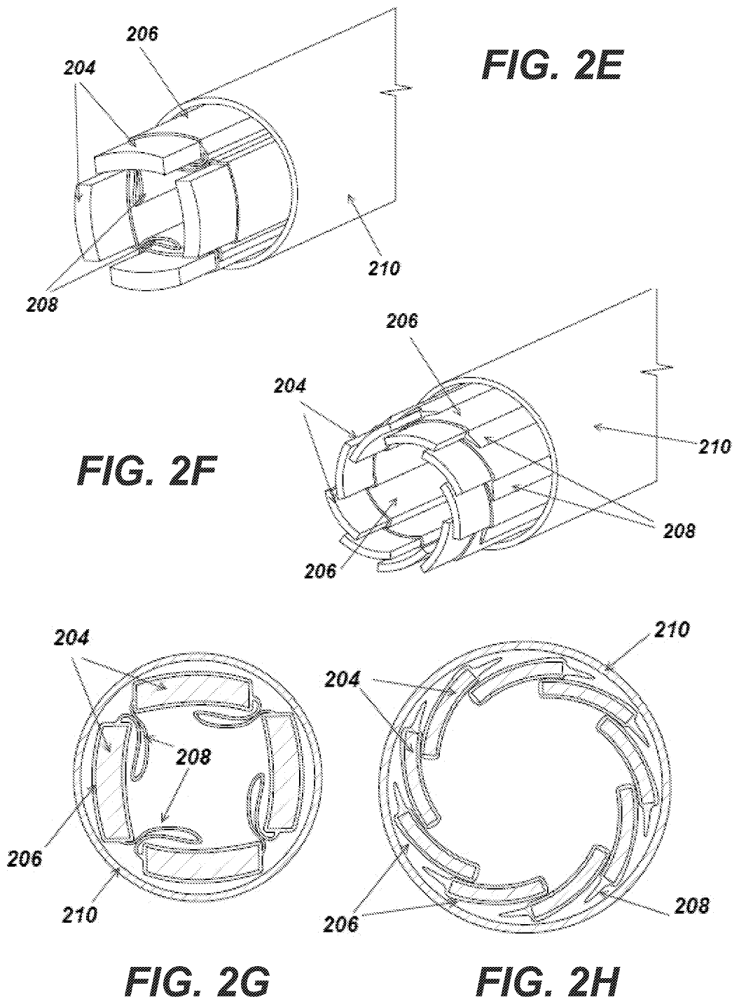

FIGS. 2A-2H show schematics of exemplary unexpanded sheath configurations in some of the disclosed herein aspects for use with a delivery apparatus such as that shown in FIG. 1. FIG. 2A shows a perspective of the unexpanded sheath 200 comprising an inner layer 202, wherein the inner layer comprises a reinforcing layer, comprising a plurality of struts 204 and a polymer layer 206 disposed around the plurality of struts, forming a plurality of encapsulated struts and a tether portion 208 of the polymer layer that circumferentially connects each of the plurality of struts to each other. In this exemplary and unlimiting aspect, for illustration purposes only, the terminal part of the plurality of struts 204 is shown not to be encapsulated within the polymer layer 206. However, it is also understood that in some exemplary and unlimiting aspects, a distal part of the sheath and/or proximal part of the sheath can comprise a reinforcing layer that extends beyond the polymer layer and is not encapsulated within the polymer layer. As can be seen further in FIG. 2B that shows a cross-sectional view of the sheath 200, the plurality of struts 204 in this exemplary aspect are positioned in random orientation when the sheath is collapsed and is unexpanded. In such aspects, the plurality of struts 204 forming the reinforcing layer can be encapsulated within the polymer layer 206 and be connected to each other by a tether portion 208 such that a randomized orientation of the struts is formed. It can be seen that each of the plurality of struts is substantially encapsulated within the polymer layer. It can be further seen that the polymer layer 206 comprises a tether portion 208 that is substantially free of the reinforcing layer.

FIGS. 2C and 2D show an additional aspect of the disclosed herein sheath. In this exemplary and unlimiting aspect, six struts 204 are arranged in such a configuration that when the sheath is in a collapsed state, the struts form an aligned organization. For example, and without limitation, and as shown in the cross-sectional view of the sheath (FIG. 2D), some of the struts 204 can form an internal shape substantially similar to a triangular, with remaining struts disposed along a circumference of the sheath, and the plurality of tether portions 208 are at least partially folded.

FIGS. 2E and 2G show an additional aspect, where four struts 204 form an aligned organization, where the struts are arranged to form a shape that is substantially similar to a square (if each of the struts has the same width) or a rectangular or a trapezoid form (if some of the struts have different width), where the tether portions are folded in between when the sheath is in the collapsed configuration. However, it is understood that the shapes shown in these figures are exemplary only, and any other desired arrangements can be formed, depending on the width of each strut, the desired diameter of the sheath, and the desired application.

Additional aspects are shown in FIGS. 2F and 2H, where the plurality of struts 204 are arranged circumferentially in a row such that at least a portion of each strut overlays at least a portion of an adjacent strut. Again, in such exemplary and unlimiting aspects, the tether portion is folded when the sheath is in a collapsed configuration. In any of the disclosed above aspects, the cross-section of the sheath in the collapsed configuration is defined by a first diameter.

Sheath, as shown in FIGS. 2A-2H can further include an outer layer 210.

The orientation of the plurality of struts forms an inner lumen through which a delivery apparatus can travel into a patient's vessel in order to deliver, remove, repair, and/or replace a prosthetic device. The disclosed sheath can also be useful for other types of minimally invasive surgery, such as any surgery requiring introducing an apparatus into a subject's vessel. For example, the disclosed sheath also can be used to introduce other types of delivery apparatus for placing various types of intraluminal devices (e.g., stents, stented grafts, etc.) into many types of vascular and non-vascular body lumens (e.g., veins, arteries, esophagus, ducts of the biliary tree, intestine, urethra, fallopian tube, other endocrine or exocrine ducts, etc.).

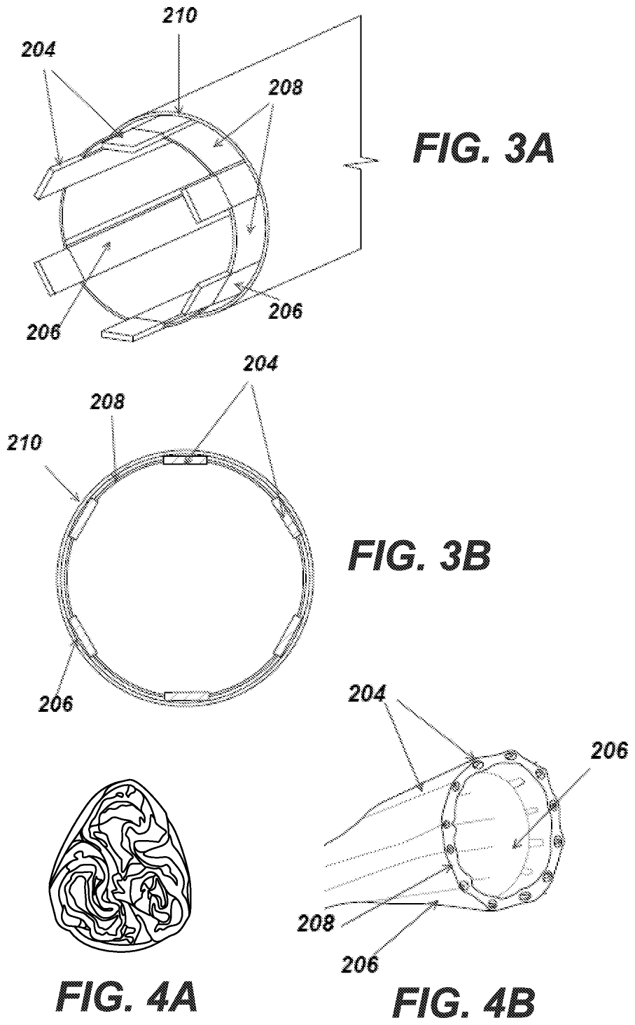

FIGS. 3A and 3B depict the exemplary sheath 200, as described in any of the aspects herein, present in an expanded configuration. It can be seen that upon expansion, the plurality of struts 204 organize in radial orientation to form a sheath having a substantially circular cross-section having a second diameter. In such aspects, the second diameter is larger than the first diameter and allows the medical device to pass through the sheath.

It is understood that the struts in any of the aspects disclosed herein can be radially spaced from each other at a predetermined length. In certain aspects, this predetermined length is defined by a length of each of the plurality of tether portions. It is understood that the predetermined length can be the same between all the struts, or it can vary. It is also understood that if the predetermined length varies between the plurality of struts, some tether portions can have a similar or a different length depending on the desired application.

In yet further aspects, the predetermined length between the plurality of struts can also be the same or vary along the length of the sheath. For example, and without limitations, the length between the plurality of struts at a proximal end of the sheath can be larger than the length between the plurality of struts at a distal end of the sheath. In yet further aspects, the length between the plurality of struts in a portion of the sheath between the proximal and the distal ends of the sheath can be different from the length between the plurality of struts at the proximal and/or distal ends of the sheath.

In still further aspects, at least a portion of the plurality of struts comprises struts that are not coupled to any adjacent struts other than through the circumferentially connecting tethering portions of the polymer layer encapsulating the plurality of struts. Such exemplary aspects can be seen in FIGS. 2A-4B.

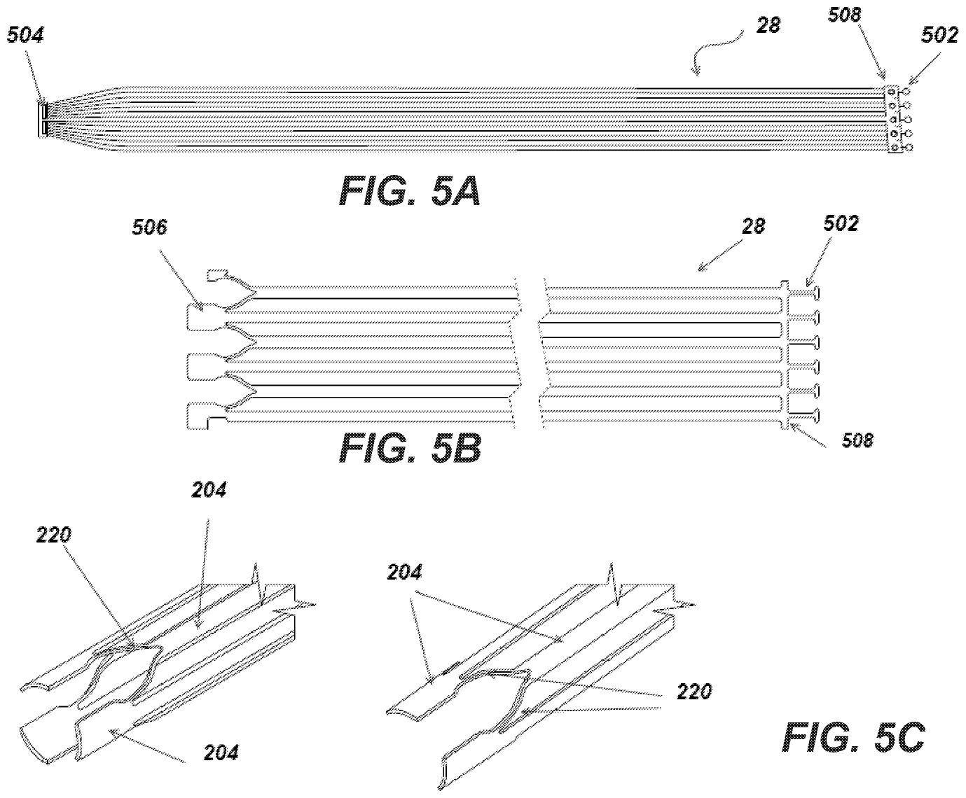

While yet in other aspects and as illustrated in FIGS. 5A-5C, the sheath can also comprise a plurality of struts having at least one strut that at least partially coupled to at least a portion of an adjacent strut by at least one bridging member. For example, as shown in FIG. 5C, a plurality of struts 204 can be connected to each other with at least one bridging member 220.

As discussed, in certain aspects, the distal end of the reinforcing layer is different from the proximal end of the reinforcing layer. For example, and as shown in FIG. 5A, the plurality of struts at the proximal end of the reinforcing layer can be coupled to each other circumferentially by at least one radially extending terminal struts 508. In yet other aspects, the struts can have a different terminal portion 506 from the rest of the strut lengths, as shown, for example, in FIG. 5B and they can also be coupled by a bridging member 220, as shown in FIG. 2C. In yet further aspects, the enclosed distal end portion can further comprise a plurality of reflow features configured to integrally connect the distal end of the reinforcing layer with a tip of the sheath. In yet other aspects, the distal end of the reinforcing layer can be tapered.

These disclosed exemplary aspects refer to the proximal end of the reinforcing layer. However, it is also understood that such radially extending struts or the struts connected by a bridging member can be located anywhere along the length of the sheath. For example, and without limitation, they can be located in a middle portion of the sheath, and/or proximal, and/or distal portion of the sheath. It is understood that the coupling can be done by any known in the art methods, for example, and without limitation, it can be done by gluing, welding, spot welding, laser welding, or any combinations thereof. In yet other aspects, the bridging member can be a separate strut or a weld, a glue, or any combination thereof.

In still further aspects, and as shown, for example, in FIGS. 5A and 5B, in some aspects, the plurality of struts at the proximal end of the reinforcing layer can also be connected to each other circumferentially by at least one radially extending terminal strut 508. In some exemplary and unlimiting aspects, the proximal end of the reinforcing layer can comprise a plurality of extension struts 502 configured to secure the reinforcing layer to a hub. In certain aspects, the plurality of extension struts can be further flared out radially to ensure a better coupling to the hub.

In some aspects, the plurality of extension struts 502 can have a T-bar shape (for example, shown in FIG. 5B), or it can have an island shape (not shown).

It is understood that struts can have any shape and configuration. In certain aspects and as shown in FIGS. 2A-2H, the struts can be flat. Yet in other aspects, as shown in FIGS. 4A and 4B, the struts can be round. The struts can also have irregular cross-sections.

As shown in FIG. 4B, for example, the plurality of struts can comprise a plurality of individual wires. These individual wires can be encapsulated with the polymer layer and be coupled to each other through the tether portions. In yet other aspects, the reinforcing layer can comprise an etched sheet forming the plurality of struts or a laser-cut sheet or tube (such, for example, and without limitation, a hypotube) that forms the plurality of struts.

In yet further aspects, the plurality of the struts can have a diameter from about 0.001″ to about 0.020″, including exemplary values of about 0.002″, about 0.003″, about 0.004″, about 0.005″, about 0.006″, about 0.007″, about 0.008″, about 0.009″, about 0.010″, about 0.011″, about 0.012″, about 0.013″, about 0.014″, about 0.015″, about 0.016″, about 0.017″, about 0.018″, and about 0.019″, It is further understood that each of the plurality of struts can have any diameter having a value between any two foregoing values. It is understood that in some aspects, each of the plurality of struts can have the same diameter, while in other aspects, the struts can have a varying diameter. In yet other aspects, the diameter of struts can be the same and uniform along the length of the sheath. While in other aspects, it can vary along any portion of the sheath.

In aspects where the struts are not round, they can be defined by a predetermined width and a predetermined thickness. In such exemplary and unlimiting aspects, the width of the struts can be in a range from about 0.001″ to about 0.100″, including exemplary values of about 0.002″, about 0.003″, about 0.004″, about 0.005″, about 0.006″, about 0.007″, about 0.008″, about 0.009″, about 0.010″, about 0.011″, about 0.012″, about 0.013″, about 0.014″, about 0.015″, about 0.016″, about 0.017″, about 0.018″, and about 0.019″, about 0.020″, about 0.025″, about 0.030″, about 0.040″, about 0.050″, about 0.060″, about 0.070″, about 0.080″, and about 0.090″. It is further understood that each of the plurality of struts can have any width having a value between any two foregoing values. It is understood that in some aspects, each of the plurality of struts can have the same width, while in other aspects, the struts can have a varying width. In yet other aspects, the width of the struts can be the same and uniform along the length of the sheath. While in other aspects, it can vary along any portion of the sheath.

In yet further aspects, the plurality of struts can have a predetermined thickness from about 0.001″ to about 0.040″, including exemplary values of about 0.002″, about 0.003″, about 0.004″, about 0.005″, about 0.006″, about 0.007″, about 0.008″, about 0.009″, about 0.010″, about 0.011″, about 0.012″, about 0.013″, about 0.014″, about 0.015″, about 0.016″, about 0.017″, about 0.018″, and about 0.019″, about 0.020″, about 0.021″, about 0.022″, about 0.023″, about 0.024″, about 0.025″, about 0.026″, about 0.027″, about 0.028″, about 0.029″, about 0.030″, about 0.031″, about 0.032″, about 0.033″, about 0.034″, about 0.035″, about 0.036″, about 0.037″, about 0.038″, and about 0.039″. It is further understood that each of the plurality of struts can have any thickness having a value between any two foregoing values. It is understood that in some aspects, each of the plurality of struts can have the same thickness, while in other aspects, the struts can have a varying thickness. In yet other aspects, the thickness of struts can be the same and uniform along the length of the sheath. While in other aspects, it can vary along any portion of the sheath.

In certain aspects, the skilled practitioner can consider the use of flat versus round or irregular cross-section struts based on their relative moments of inertia about a given bend axis and optimization of stiffness during delivery, payload insertion, and withdrawal.

It is understood that aspects disclosed herein can comprise a reinforcing layer formed of any material that can provide for the desired stiffness and is capable to provide reinforcing properties to the sheath. In certain aspects, the reinforcing layer can comprise a metal or a polymer.

In such exemplary aspects, the polymer can be any polymer. In yet other aspects, the polymer can comprise any hard plastic. In still further aspects, the polymer can comprise PEEK, nylon, or a combination thereof. In still further aspects, the polymer can have any modulus that provides for the desired stiffness profile. In certain aspects, the polymer can exhibit modulus from about 1 GPa to about 10 GPa, including exemplary values of about 2 GPa, about 3 GPa, about 4 GPa, about 5 GPa, about 5 GPa, about 6 GPa, about 7 GPa, about 8 GPa, and about 9 GPa.

In still further exemplary and unlimiting aspects, when the plurality of struts comprise a polymer, the polymer can have a Shore hardness from about 10 A to about 90 A or from about 10 D to about 90 D. In certain aspects, the polymer can have a Shore hardness from about 10 A to about 90 A, including exemplary values of about 20 A, about 30 A, about 40 A, about 50 A, about 60 A, about 70 A, and about 90 A. In yet other aspects, the polymer can have a Shore hardness from about 10 D to about 90 D, including exemplary values of about 20 D, about 30 D, about 40 D, about 50 D, about 60 D, about 70 D, and about 90 D.

In yet still, further aspects, the plurality of struts can comprise a metal. In such exemplary and unlimiting aspects, the metal can be titanium metal, nitinol, cobalt-chromium alloys, stainless steel, or any combinations or alloys thereof. In certain aspects, and as described herein, when the reinforcing layer comprises the metal, the metal can exhibit a modulus from about 20 GPa to about 250 GPa, including exemplary values of about 50 GPa, about 60 GPa, about 70 GPa, about 80 GPa, about 90 GPa, about 100 GPa, about 110 GPa, about 120 GPa, about 130 GPa, about 140 GPa, about 150 GPa, about 160 GPa, about 170 GPa, about 180 GPa, about 190 GPa, about 200 GPa, about 210 GPa, about 220 GPa, about 230 GPa, and about 240 GPa to achieve the desired predetermined stiffness profile.

In yet other aspects, the sheath disclosed herein can be configured to receive an introducer configured to reinforce a radial orientation of the plurality of encapsulated struts upon introducing the sheath into a body. The introducer can comprise at least a partially grooved surface configured to match the radial orientation of the plurality of encapsulated struts. It is understood that the introducer can be used to help organize the plurality of struts to achieve orientation that would allow passage of the medical device.

In still further aspects, the polymer layer disclosed herein can comprise any polymers known in the art and suitable for the desired application. It is understood that in some aspects, the polymer layer is lubricious. In such exemplary and unlimiting aspects, the polymer layer has a friction coefficient of about 0.1 or less, of about 0.09 or less, about 0.08 or less, about 0.07 or less, about 0.05 or less, about 0.04 or less, about 0.03 or less, about 0.02 or less, or about 0.01 or less.

In yet further aspects, the polymer layer is substantially not stretchable. In certain aspects, the polymer layer can comprise ePTFE, PTFE, polyethylene, polyvinylidene fluoride, an ultra-high molecular weight polyethylene (UHMWPE) (for example, Dyneema®) and combinations thereof.

In still further aspects, the polymer layer can comprise one or more sublayers. In some aspects, if one or more sublayers are present, each sublayer can comprise the same or different polymer. In yet further aspects, the encapsulation of the reinforcing layer with the polymer layer can be done by a sintering process.

In still further aspects, a combined thickness of the polymer layer and the reinforcing layer is from about 0.003″ to about 0.040″, including exemplary values of about 0.004″, about 0.005″, about 0.006″, about 0.007″, about 0.008″, about 0.009″, about 0.010″, about 0.011″, about 0.012″, about 0.013″, about 0.014″, about 0.015″, about 0.016″, about 0.017″, about 0.018″, and about 0.019″, about 0.020″, about 0.021″, about 0.022″, about 0.023″, about 0.024″, about 0.025″, about 0.026″, about 0.027″, about 0.028″, about 0.029″, about 0.030″, about 0.031″, about 0.032″, about 0.033″, about 0.034″, about 0.035″, about 0.036″, about 0.037″, about 0.038″, and about 0.039″. In yet other aspects, the combined thickness of the polymer layer and the reinforcing layer can be any value between any two foregoing values. For example, the combined thickness can be from about 10 taus to about 40 taus, or from about 15 taus to about 35 taus.

In still further aspects, a thickness of the tether portion can be from less than about 0.001″ to about 0.030″ including exemplary values of about 0.0001″, about 0.0002″, about 0.0003″, about 0.0004″, about 0.0005″, about 0.0006″, about 0.0007″, about 0.0008″, about 0.0009″, about 0.001″, about 0.002″, about 0.003″, about 0.004″, about 0.005″, about 0.006″, about 0.007″, about 0.008″, about 0.009″, about 0.010″, about 0.011″, about 0.012″, about 0.013″, about 0.014″, about 0.015″, about 0.016″, about 0.017″, about 0.018″, and about 0.019″, about 0.020″, about 0.021″, about 0.022″, about 0.023″, about 0.024″, about 0.025″, about 0.026″, about 0.027″, about 0.028″, about 0.029″, about 0.030″, about 0.031″, about 0.032″, about 0.033″, about 0.034″, about 0.035″, about 0.036″, about 0.037″, about 0.038″, and about 0.039″. In yet other aspects, the thickness of each of the plurality of tether portions can be any value between any two foregoing values. For example, the thickness of each of the plurality of tether portions can be from less than 0.001″ to about 0.040″, or from about 0.005″ to about 0.015″.

In still further aspects and as disclosed herein, the sheath can also comprise an outer layer. In certain aspects, the outer layer can be an elastomeric material. In yet further aspects, the outer layer can extend all the way from the proximal end of the sheath to the distal end of the sheath. While in some exemplary and unlimiting aspects, the outer layer can extend only partway from the proximal end of the sheath. The outer cover is positioned to surround the entire circumference of the inner layer and comprises any known in the art pliable, elastic material(s) that can expand and contract. In some aspects, the outer layer can have a high expansion ratio.

The outer layer comprising an elastomeric material can, in some aspects, provide hemostasis (e.g., prevent blood loss during implantation of the prosthetic device). For example, the outer layer 210 can be sized or configured to form a seal with the patient's artery when inserted, such that blood is substantially prevented from flowing between the outer layer 210 and the vessel wall. The outer layer 210 can be inserted such that it passes the arteriotomy.

In such aspects, the elastomeric outer layer can comprise any suitable materials, such as any suitable heat shrink materials. In yet other aspects, the outer layer comprises a compound comprising a heat shrinking material, a polyether block amide, a polyurethane, silicone, polyisoprene, or any combinations thereof present up to 100 wt %, including exemplary values of about 1%, about 10%, about 20%, about 30%, about 40%, about 50%, about 60%, about 70%, about 80%, about 90%, and 100% based on a total weight of the compound. In yet further exemplary aspects, the outer layer comprises PEBAX®.

In still further aspects, the outer layer can also comprise a styrene-based elastomer, polyurethane, latex, copolymers thereof, blends thereof, or co-extrudates of thereof. In certain and unlimiting aspects, the elastomeric polymer can comprise polyether block ester copolymer, polyesters, polyvinyl chloride, thermoset silicone, poly-isoprene rubbers, polyolefin, other medical grade polymers, or combinations thereof. In yet further aspects, the elastomeric polymer described herein can have any useful additives. In certain aspects, the elastomeric polymers can comprise at least one friction reduction additive. In some exemplary aspects, the friction reduction additives can comprise, for example, BaSO4, ProPell™, PTFE, any combination thereof, and the like. It is understood that this list of friction reduction additives is not limiting, and any known in the art friction reductions additives can be utilized.

In still further aspects, the outer layer can have a Shore hardness of from about 25 Durometer to about 90 Durometer, including exemplary values of about 30 Durometer, about 40 Durometer, about 45 Durometer, about 50 Durometer, about 55 Durometer, about 60 Durometer, about 65 Durometer, about 70 Durometer, about 75 Durometer, about 80 Durometer, and about 85 Durometer.

It is understood that in some aspects, the outer layer can have the same Shore hardness along the length of the sheath. In yet other aspects, the Shore hardness of the polymer layer can vary along the length of the sheath. For example, and without limitation, disclosed herein are aspects where a durometer of the outer layer at the proximal end of the sheath can be different from a durometer of the outer layer at the distal end of the sheath.

In still further aspects, the outer layer can comprise various polymer layers. In some aspects, the outer layer can comprise the first polymer layer, having a first compound composition comprising from greater than 0 wt % to less than 100 wt %, including exemplary values of about 0.01 wt %, about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, and about 99.9 wt % of a polymer comprising a polyether block amide, a polyurethane, or any combinations thereof.

In still further aspects, the first compound composition can comprise from greater than about 35 wt % to less than about 80 wt %, including exemplary values of about 40 wt %, about 45 wt %, about 50 wt %, about 55 wt %, about 60 wt %, about 65 wt %, about 70 wt %, and about 75 wt % of a polymer comprising a polyether block amide, a polyurethane, or any combinations thereof.

In certain aspects, the polymer in the first compound composition comprises a polyether block amide. In such exemplary aspects, the polyether block amide can comprise PEBAX® from Arkema. In yet further aspects, the polymer can comprise polyurethane, for example, NEUSoft®. While in still further aspects, the polymer can compromise a combination of the polyether block amide, such as, for example, PEBAX® and polyurethane. It is further understood that if the mixture of the polymers is present, such a mixture can comprise each component in any amount relative to another component to provide the desired polymer falling within the disclosed above range.

In still further aspects, the first compound composition can comprise less than about 65 wt % of an inorganic filler based on a total weight of the first compound composition, including exemplary values of less than about 60 wt %, less than about 55 wt %, less than about 50 wt %, less than about 45 wt %, less than about 40 wt %, less than about 35 wt %, less than about 30 wt %, less than about 25 wt %, less than about 20 wt %, less than about 15 wt %, less than about 10 wt %, less than about 5 wt %, and less than about 1 wt % of the inorganic filler.

In yet further aspects, the inorganic filler can be present in an amount of at least about 1 wt %, at least about 2 wt %, at least about 5 wt %, at least about 10 wt %, at least about 15 wt %, at least about 20 wt %, at least about 25 wt %, at least about 30 wt %, at least about 35 wt %, at least about 40 wt %, at least about 45 wt %, at least about 50 wt %, or at least about 55 wt %.

In still further aspects, the inorganic filler can comprise any inorganic materials that can be used as a filler and are acceptable for the desired application. In certain exemplary and unlimiting aspects, the inorganic filler can comprise bismuth oxychloride, barium sulfate, bismuth subcarbonate, calcium carbonate, aluminum trihydrate, barite, kaolin clay, limestone, or any combinations thereof. Again it is understood that the inorganic filler can comprise a combination of the various fillers. In such exemplary aspects, an amount of each filler in the combination can be in any range to provide a final combination that falls within the disclosed above range.

In still further aspects, the first compound composition can comprise up to about 20 wt % of a solid lubricant filler based on a total weight of the first compound composition, including exemplary values of about 0.01 wt %, about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, and about 19.9 wt %. In yet further aspects, the solid lubricant filler can be present up to about 20 wt %, up to about 15 wt %, or up to about 10 wt % based on a total weight of the first compound composition.

In still further aspects, the solid lubricant filler can comprise any additive that is known to reduce friction and behave as a lubricant. In such exemplary and unlimiting aspects, the solid lubricant filler can comprise one or more of graphene, reduced graphene oxide, carbon black, boron nitride, silicones, talc, polytetrafluorethylene (PTFE), fluorinated ethylene propylene, and the like. In still further aspects, the solid lubricant comprises a PTFE filler. In yet further aspects, the PTFE filler is a powder.

In still further aspects, the first compound composition can further comprise at least one tackiness reducing compound. Any compounds known in the art as capable of reducing the tackiness of the polymer composition can be considered and used for the purpose of this disclosure. In yet further exemplary and unlimiting aspects, the at least one tackiness reducing compound comprises ProPell™ from Foster Corporation.

In certain aspects, the at least one tackiness reducing compound is present in an amount from 0 wt % to about 20 wt %, including exemplary values of about 0.01 wt %, about 0.05 wt %, about 0.1 wt %, about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, and about 19 wt % based on a total weight of the first compound composition. In still further aspects, the at least one tackiness reducing compound is present in any amount having a value between any two foregoing values. For example, and without limitation, the at least one tackiness reducing compound can be present in an amount from about 1 wt % to about 5 wt %, or from about 5 wt % to about 10 wt % based on a total weight of the first compound composition.

Yet, in other aspects, two or more polymer layers can be present in the outer layer. In such exemplary aspects, the outer layer can comprise at least one second polymer layer comprising a second compound composition. It is understood that the second compound composition can be the same or different. It can comprise the same or different inorganic fillers, solid lubricants, and optionally tackiness reducing agents. In yet other aspects, the second compound composition does not comprise inorganic fillers, solid lubricants, and/or tackiness reducing agents. In still further aspects, each layer present in the outer layer can have the same or different Shore hardness.

It is further understood that in certain aspects, the first polymer in the first compound composition can be the same as the second polymer in the second compound composition. Yet, in other aspects, the first polymer in the first compound composition is different from the second polymer in the second compound composition. In yet further aspects, the second polymer layer composition comprises PEBAX®. While in further aspects, the second polymer layer composition can comprise polyurethane, for example, NEUSoft® from PolyOne.

In still further aspects, and as disclosed herein, the outer layer has a predetermined thickness, and wherein at least about 10%, at least about 20%, at least about 30%, at least about 40%, at least about 50%, at least about 60%, at least about 70%, at least about 80%, at least about 90%, or 100% of the predetermined thickness comprises the first and/or the second compound composition comprising the first and/or the second polymer having a Shore D equal to or lower than about 30 D.

The outer layer can have a thickness ranging from, for example, about 0.001″ to about 0.010″, including exemplary values of about 0.002″, about 0.003″, about 0.004″, about 0.005″, about 0.006″, about 0.007″, about 0.008″, and about 0.009″.

In still further aspects, the thickness of the outer layer can vary along the length of the sheath. Yet, in further aspects, the thickness of the outer layer is greater at the proximal end.

In still further aspects, if, for example, two polymer layers are present in the outer layer, each of the polymer layers can have the same thickness. While in other aspects, the first polymer layer and the second polymer layer have different thicknesses. For example, in some aspects, the first polymer layer has a thickness of about 0.001″ to about 0.003″, including exemplary values of about 0.0011″, about 0.0012″, about 0.0013″, about 0.0014″, about 0.0015″, about 0.0016″, about 0.0017″, about 0.0018″, about 0.0019″, about 0.002″, about 0.0021″, about 0.0022″, about 0.0023″, about 0.0024″, about 0.0025″, about 0.0026″, about 0.0027″, about 0.0028″, and about 0.0029″. Yet still, in further aspects, the second polymer layer can have a thickness of about 0.002″ to about 0.004″, including exemplary values of about 0.0021″, about 0.0022″, about 0.0023″, about 0.0024″, about 0.0025″, about 0.0026″, about 0.0027″, about 0.0028″, about 0.0029″, about 0.003″, about 0.0031″, about 0.0032″, about 0.0033″, about 0.0034″, about 0.0035″, about 0.0036″, about 0.0037″, about 0.0038″, about 0.0039″.

In still further aspects, the thickness of the outer layer is greater at the proximal end. While in other aspects, the thickness of the outer layer is smaller at the distal end than the thickness of the outer layer at the proximal end.

In still further aspects, the outer layer can be extruded. In the aspects where the first and the second polymer layers are present, such polymer layers can be co-extruded. In still further aspects, the first polymer layer can be substantially bonded to the second polymer layer. In such exemplary aspects, the first polymer layer substantially does not delaminate from the second polymer layer. It is understood that in some aspects, the bonding can be physical or chemical, or any other type known in the art.

In yet further aspects, the outer layer is configured to apply an inward radial force on the sheath, biasing the sheath toward the unexpanded state.

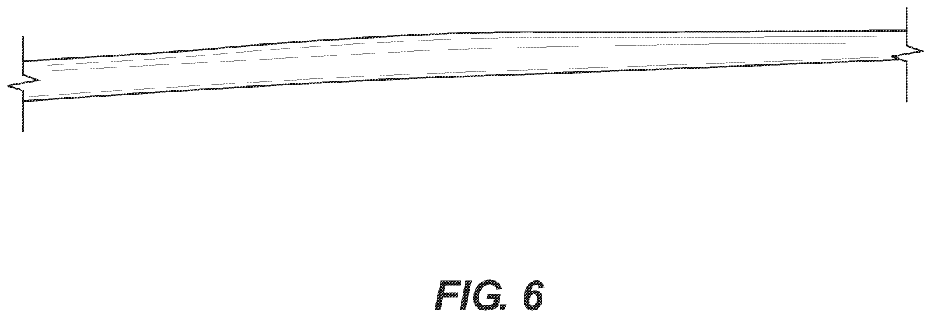

In certain aspects, the first diameter (unexpanded diameter) can be anywhere between about 10 Fr to about 16 Fr, including exemplary values of about 10.5 Fr, about 11 Fr, about 12 Fr, 12.5 Fr, about 13 Fr, about 13.5 Fr, about 14 Fr, about 14.5 Fr, about 15 Fr, and about 15.5 Fr. In yet other aspects, upon passage of the medical device the lumen can be expanded locally by radially arranging and orienting struts to arrive at the second diameter from about 20 Fr to about 26 Fr, including exemplary values of about 20.5 Fr, about 21 Fr, about 21.5 Fr, about 22 Fr, about 22.5 Fr, about 23 Fr, about 23.5 Fr, about 24 Fr, about 24.5 Fr, about 25 Fr, and about 25.5 Fr. After the passing of the device, the inner layer returns to its initial configuration having a diameter that is substantially identical to the first diameter. It is understood, and as described above, the outer layer can assist in the contraction of the inner layer by applying an inward radial force on the sheath. FIG. 6 shows an exemplary photograph of a locally expanded exemplary sheath, according to some aspects disclosed herein.

Aspects of the disclosed sheath can expand to an expanded outer diameter that is from about 10% greater than the original unexpanded outer diameter to about 100% greater than the original unexpanded outer diameter, including exemplary values of about 15% greater, about 20% greater, about 25% greater, about 30% greater, about 35% greater, about 40% greater, about 45% greater, about 50% greater, about 55% greater, about 60% greater, about 65% greater, about 70% greater, about 75% greater, about 80% greater, about 85% greater, about 90% greater, and about 95% greater than the original unexpanded outer diameter.

It is understood, and as described above, the disclosed sheath can expand from its rest position. The expansion of the disclosed sheath can result in a second diameter d₂ that is from about 10% or less to about 430% or more than the first diameter d₁. In certain aspects, expansion of the sheath can result in expansion of the first diameter dr to about 10% or less, to about 9% or less, to about 8% or less, to about 7% or less, to about 6% or less, to about 5% or less, to about 4% or less, to about 3% or less, to about 2% or less, to about 1% or less. In yet other aspects, expansion of the disclosed sheath can result in expansion of the first diameter dr to about 10% or more, about 20% or more, about 30% or more, about 40% or more, about 50% or more, about 60% or more, about 70% or more, about 80% or more, about 90% or more, about 100% or more, about 125% or more, about 150% or more, about 175% or more, about 200% or more, about 225% or more, or about 250% or more.

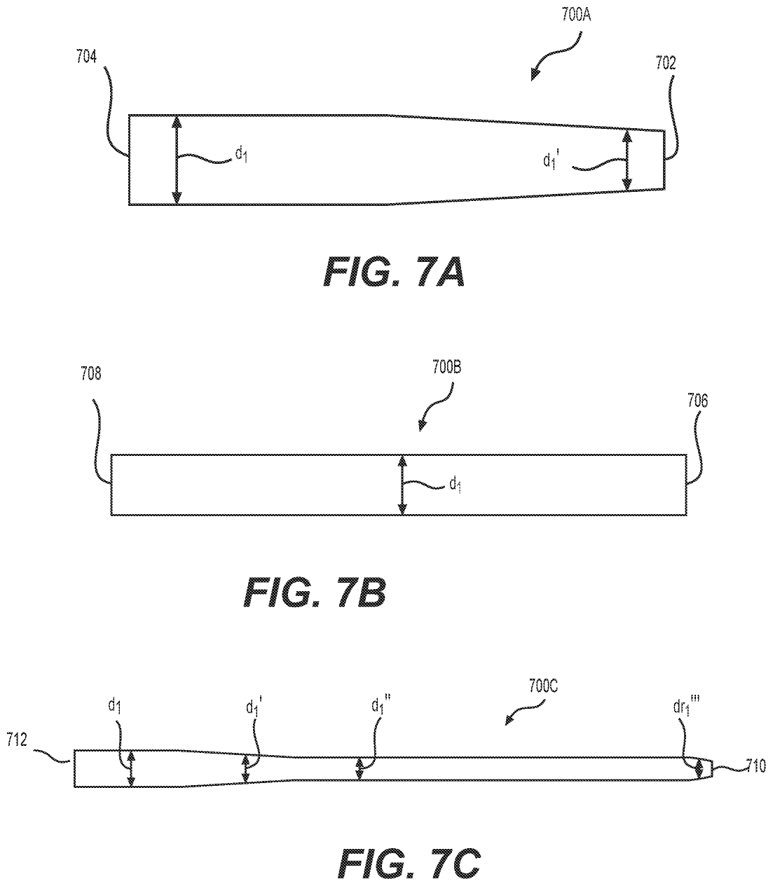

It is understood that the diameter of the sheath can be the same or different along a longitudinal axis of the sheath. The first diameter d₁ of the lumen can vary depending on the application and size of the delivery apparatus and prosthetic device. FIG. 7A-C show various configurations and shapes of the sheath. It is understood that in some aspects, and as shown in FIG. 7B, the first diameter d₁ is substantially uniform along the longitudinal axis of the lumen without changing from the proximal end 708 to the distal end 706. In yet other aspects, and as shown in FIGS. 7A and 7C, the first diameter d₁ can vary along the longitudinal axis (for example, d₁ and d₁′ in FIG. 7A, or d₁, d₁′, d₁″, and d₁″, as shown in FIG. 7C) of the lumen. In certain aspects, the first diameter dᵣ₁ at the proximal end 704 or 712 is larger than the first diameter d₁′ as shown in FIG. 7A and FIG. 7C or d₁′″ as shown in FIG. 7C at the distal end 702 or 710 d₁.

In yet further aspects, where the outer layer conforms to the shape of the inner liner, the outer diameter dₒ (not shown) comprises the overall diameter of the inner layer and the outer layer. In such aspects, the outer diameter dₒ is defined by the specific application of the sheath. Similar to the rest diameter d₁, the outer diameter dₒ of the unexpended sheath disclosed herein can be substantially uniform (constant) along the longitudinal axis of the lumen without changing from the proximal end to the distal end (not shown). In alternative aspects, the original unexpanded outer diameter dₒ of the disclosed sheath, similarly to the first diameter d₁, can decrease from the proximal end to the distal end. In some aspects, and similarly to the first diameter d₁, the original unexpanded outer diameter can decrease along a gradient, from the proximal end to the distal end; or it can incrementally step down along the length of the sheath having the largest original unexpanded outer diameter is near do the proximal end, and the smallest original unexpanded outer diameter dₒ is near the distal end.

Different sheaths can be provided with different first diameter d₁, and outer diameters do, depending on the size requirements of the delivery apparatus for various applications. Additionally, some aspects can provide more or less expansion depending on the particular design parameters, the materials, and/or configurations used. In some aspects, the outer diameter dₒ of the sheath gradually decreases from the proximal end of the sheath to the distal end of the sheath. For example, in one aspect, the outer diameter dₒ can gradually decrease from about 26 Fr at the proximal end to about 18 Fr at the distal end. The diameter dₒ of the sheath can transition gradually across substantially the entire length of the sheath. In other aspects, the transition or reduction of the diameter of the sheath can occur only along a portion of the length of the sheath. For example, the transition can occur along a length from the proximal end to the distal end, where the length can range from about 0.5 inches to about the entire length of the sheath, including any values between any two foregoing values. In yet further aspects, the do is minimal and constant along the section of the sheath that passes through the vasculature. In such aspects, the tapered section is about 4″ or less at the proximal side of the sheath.

In still further aspects, a tie layer can be present between the outer layer and the inner layer of the sheath. In certain and unlimiting aspects, a lubricant can be applied between the overlapping portions of the encapsulated reinforcing layer to allow easier expansion during the introduction of the medical device. In such exemplary aspects, the lubricant can comprise Christo Lube supplied by ECL or MED10/6670 supplied by Nusil.

In still further aspects, the sheath, as disclosed herein, is substantially kink-resistant. Yet, in other aspects, the sheath can exhibit at least a 10% reduction, at least about 15% reduction, at least 20% reduction, at least about 25% reduction, at least about 30% reduction, at least about 35% reduction, at least about 40% reduction, at least about 45% reduction, or at least about 50% reduction in an insertion force when compared with an insertion force of a commercially available sheath or any sheath that does not comprise the disclosed herein structure. In still further aspects, the sheath of the instant disclosure can comprise a hemostasis valve inside the lumen of the sheath, at or near the proximal end of the sheath (not shown). Additionally, the exemplary sheaths disclosed herein can comprise a soft tip at the distal end of the sheath (not shown). Such a soft tip can be provided with a lower hardness than the other portions of the sheath. In some aspects, the soft tip can have a Shore hardness from about 25 D to about 40 D, including exemplary values of about 26 D, about 27 D, about 28 D, about 29 D, about 30 D, about 31 D, about 32 D, about 33 D, about 34 D, about 35 D, about 36 D, about 37 D, about 38 D, and about 39 D. In yet other aspects, the soft tip can have a Shore hardness from about 25 A to about 40 A, including exemplary values of about 26 A, about 27 A, about 28 A, about 29 A, about 30 A, about 31 A, about 32 A, about 33 A, about 34 A, about 35 A, about 36 A, about 37 A, about 38 A, and about 39 A.

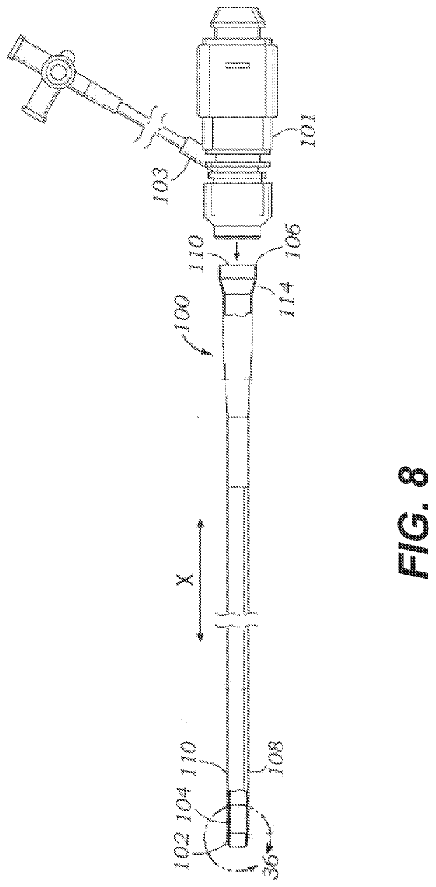

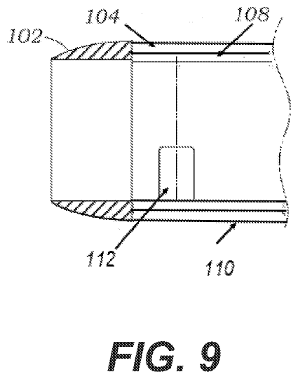

FIGS. 8 and 9 illustrate an expandable sheath 100 according to the present disclosure, which can be used with a delivery apparatus for delivering a prosthetic device, such as a tissue heart valve into a patient. In general, the delivery apparatus can include a steerable guide catheter (also referred to as a flex catheter), a balloon catheter extending through the guide catheter, and a nose catheter extending through the balloon catheter (e.g., as depicted in FIG. 1). The guide catheter, the balloon catheter, and the nose catheter can be adapted to slide longitudinally relative to each other to facilitate delivery and positioning of the valve at an implantation site in a patient's body. However, it should be noted that the sheath 100 can be used with any type of elongated delivery apparatus used for implanting balloon-expandable prosthetic valves, self-expanding prosthetic valves, and other prosthetic devices. Generally, sheath 100 can be inserted into a vessel (e.g., the femoral or iliac arteries) by passing through the skin of a patient, such that a soft tip portion 102 at the distal end 104 of the sheath 100 is inserted into the vessel. The sheath 100 can also include a proximal flared end portion 114 to facilitate mating with an introducer housing 101 (or a hub) and catheters mentioned above (e.g., the proximal flared end portion 114 can provide a compression fit over the housing tip and/or the proximal flared end portion 114 can be secured to the housing (hub) 101 via a nut or other fastening device or by bonding the proximal end of the sheath to the housing). The reinforcing layer, as described above, can comprise a plurality of extension struts 502 (FIG. 5B) that allow for better mating with the hub. The introducer housing 101 can house one or more valves that form a seal around the outer surface of the delivery apparatus once inserted through the housing, as known in the art. The delivery apparatus can be inserted into and through the sheath 100, allowing the prosthetic device to be advanced through the patient's vasculature and implanted within the patient.

In exemplary aspects, the sheath 100 comprises the disclosed herein inner liner 108 and an outer layer 110 disposed around the inner liner 108. The inner liner 108 (having a reinforcing layer encapsulated into a polymer layer) defines a lumen having the first diameter d₁ through which a delivery apparatus can travel into a patient's vessel in order to deliver, remove, repair, and/or replace a prosthetic device, moving in a direction along the longitudinal axis X. As the prosthetic device passes through the sheath 100, the sheath locally expands from the first diameter d₁ to the expanded second diameter d₂ to accommodate the prosthetic device. After the prosthetic device passes through a particular location of the sheath 100, each successive expanded portion or segment of the sheath 100 at least partially returns to the first diameter d₁. In this manner, the sheath 100 can be considered self-expanding in that it does not require the use of a balloon, dilator, and/or obturator to expand.

The inner liner 108 and outer layer 110, as shown herein, can comprise any materials disclosed above.

Additionally, some aspects of a sheath 100 can include an exterior hydrophilic coating on the outer surface of the outer layer 110. Such a hydrophilic coating can facilitate insertion of the sheath 100 into a patient's vessel. Examples of suitable hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N. V, Heerlen, the Netherlands), as well as other hydrophilic coatings (e.g., PTFE, polyethylene, polyvinylidene fluoride), are also suitable for use with the sheath 100.

FIG. 9 shows a portion of the exemplary sheath along a line 36 in FIG. 8. In such exemplary aspects, a soft tip 102 can be attached to the distal end 104 portion of the sheath 100. Best seen in FIG. 9, the soft tip portion 102 can comprise, in some aspects, low density polyethylene (LDPE) and can be configured to minimize trauma or damage to the patient's vessels as the sheath is navigated through the vasculature. For example, in some aspects, the soft tip portion 102 can be slightly tapered to facilitate passage through the vessels. The soft tip portion 102 can be secured to the distal end 104 of the sheath 100, such as by thermally bonding the soft tip portion 102 to the inner and outer layers of the sheath 100. As disclosed above, to facilitate such mating, the reinforcing layer can comprise a plurality of reflow features 504 (FIG. 5A) configured to integrally connect the distal end of the reinforcing layer with a tip of the sheath. Such a soft tip portion 102 can be provided with a lower hardness than the other portions of the sheath 100. In some aspects, the soft tip 102 can have a Shore hardness from about 25 A to about 40 A, including exemplary values of about 28 A, about 30 A, about 32 A, about 35 A, and about 38 A. It is further understood that Shore hardness can have any value between any two foregoing values. In yet other aspects, the soft tip 102 can have a Shore hardness from about 25 D to about 40 D, including exemplary values of about 28 D, about 30 D, about 32 D, about 35 D, and about 38 D. The tip portion 102 is configured to be radially expandable to allow a prosthetic device to pass through the distal opening of the sheath 100.

As shown in FIG. 9, the sheath 100 can optionally include at least one radiopaque filler or marker, such as a discontinuous or C-shaped, band 112 positioned near the distal end 104 of the sheath 100. The marker/band 112 can be associated with the inner liner 108 and/or outer layer 110 of the sheath 100. Such a radiopaque tip marker can comprise materials such as those suitable for the radiopaque filler, platinum, iridium, platinum/iridium alloys, stainless steel, other biocompatible metals, or combinations thereof. Suitable materials for use as a radiopaque filler or marker include, for example, barium sulfite, bismuth trioxide, titanium dioxide, bismuth subcarbonate, or combinations thereof. The radiopaque filler can be mixed with or embedded in the layer of the elastomeric polymer used to form the outer layer and can comprise from about 5% to about 45% by weight of the outer layer, including exemplary values of about 10%, about 15%, about 20%, about 25%, about 30%, about 35%, and about 40% by weight of the outer polymeric tubular layer. The more or less radiopaque material can be used in some aspects, depending on the particular application.

The disclosed herein sheath can be configured such that it locally expands at a particular location corresponding to the location of the medical device along the length of the lumen and then locally contracts once the medical device has passed that particular location. Thus, a bulge may be visible (FIG. 6), traveling longitudinally along the length of the sheath as a medical device is introduced through the sheath, representing continuous local expansion and contraction as the device travels the length of the sheath. In some aspects, each segment of the sheath can locally contract after removal of any radial outward (insertion) force such that it regains the original resting (first) diameter of lumen d₁.

In some aspects, each segment of the sheath can locally contract after removal of any radial outward force such that it at least partially returns to the original resting (first) diameter of lumen d₁.

It is further understood that the inventive sheaths can allow for more symmetric expansion when compared with currently available and commercial sheaths, which provides redundancy in the expansion direction to accommodate the path of least resistance based on the specific anatomy and other boundary conditions.

In still further aspects, an absence of radial members in the exemplary sheath can (other than bridging members when present) allow a safer pass of the crimped valve as it advances through the sheath.

It is understood that an expansion of the sheaths disclosed herein can be facilitated by small movements of multiple elements (the plurality of struts), which create multiple small and flat frictional surfaces rather than one large “crescent” shaped surface in which the expansion forces induced by the advancing delivery system may increase the friction by increasing the normal forces on the frictional surfaces. The expansion can also be facilitated by small movements of multiple elements (the plurality of struts) that distribute the elongation of the outer layer along its circumference, which minimizes the elongation stress.

Methods

The aspects of the present disclosure also relate to a method of making a sheath comprising: a) providing an inner layer comprising: i) a reinforcing layer comprising a plurality of struts axially extending from the proximal end to the distal end and circumferentially arranged in a predetermined pattern; and ii) a polymer layer disposed around the plurality of struts to form a plurality of encapsulated struts; and wherein the polymer layer further comprises a plurality of tether portions circumferentially connecting each of the plurality of struts to each other; b) disposing an outer layer around the inner layer such that the outer layer is configured to maintain a general shape of the sheath; such that when the formed sheath is in the unexpanded state the plurality of encapsulated struts are configured to collapse to a randomized or an aligned organization to form a sheath defining a cross-section having a first diameter; wherein the plurality of tether portions at least partially folded between two adjacent struts of the plurality of encapsulated struts; and when the sheath is in the expanded state the plurality of encapsulated struts and the plurality of tether portions together configured to organize to form a sheath having a substantially circular cross-section having a second diameter; wherein the second dimeter is larger than the first diameter; and wherein the formed sheath is configured to resist axial elongational of the sheath such that a length of the sheath remains substantially constant.

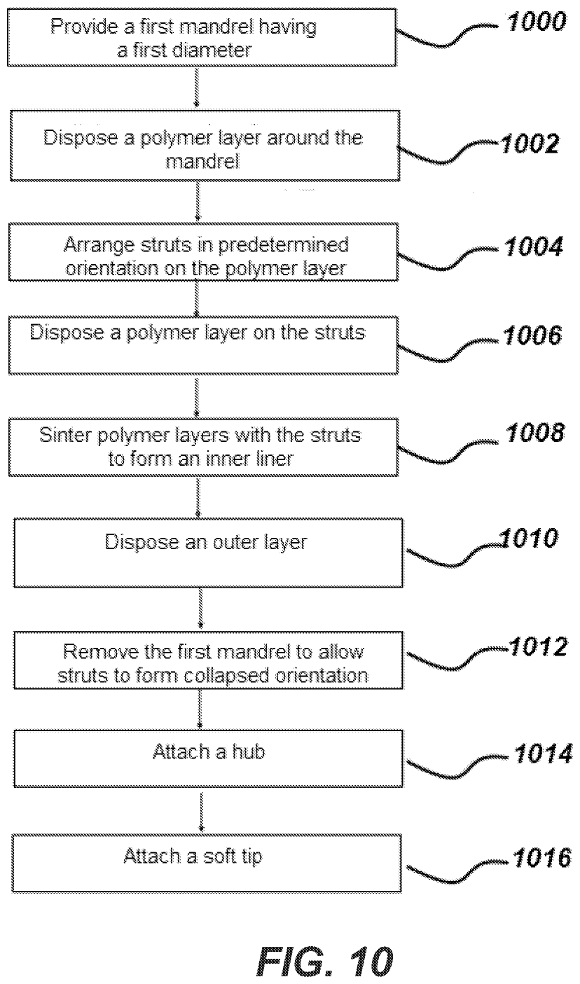

Various methods can be used to produce the sheaths discussed above and below throughout the present disclosure. For example, FIG. 10 exemplify block diagrams of one of the exemplary methods of producing the sheath in various aspects.

In still further aspects, one or more mandrels can be provided (step 1000 in FIG. 10). The mandrel can be provided with an exterior coating, such as a Teflon® coating, and the mandrel's diameter can be predetermined based on the desired rest diameter dr of the resulting sheath. A polymer layer is then disposed around the mandrel, as shown in step 1002. The struts are then arranged in a predetermined orientation on the polymer layer (step 1004), and an additional layer of the polymer layer is applied thereon (step 1006). The configuration is sintered to ensure that the struts are encapsulated within the polymer layer, and the tether portions are formed (step 1008). The outer layer is then disposed on top of the encapsulated reinforcing layer (step 1010). The mandrel is then removed (step 1012) to allow the struts to form the collapsed orientation of the sheath. In further steps, a hub (step 1014) and a soft tip (step 1016) can be further attached.

It is understood that any of the disclosed above materials can be used to form any described layer.

In still further aspects, the disclosed herein methods can comprise a step of disposing a hydrophilic coating layer on the outer surface of the layer of the elastomeric polymer. Any disclosed herein hydrophilic coating can be used.

Sheaths of the present disclosure can be used with various methods of introducing a prosthetic device into a patient's vasculature. One such method comprises positioning an expandable sheath in a patient's vessel, passing a device through the introducer sheath, which causes a portion of the sheath surrounding the device to expand and accommodate the profile of the device, and automatically retracting the expanded portion of the sheath to its original size after the device has passed through the expanded portion.stop In some methods, the expandable sheath can be sutured to the patient's skin at the insertion site so that once the sheath is inserted at the proper distance within the patient's vasculature, it does not move once the implantable device starts to travel through the sheath.

The introducer can then be removed, and a medical device, such as a transcatheter heart valve, can be inserted into the sheath, in some instances, using a loader. Such methods can additionally comprise placing the tissue heart valve in a crimped state on the distal end portion of an elongated delivery apparatus and inserting the elongated delivery device with the crimped valve into and through the expandable sheath. Next, the delivery apparatus can be advanced through the patient's vasculature to the treatment site, where the valve can be implanted.

Typically, the medical device has a greater outer diameter than the diameter of the sheath in its original configuration. The medical device can be advanced through the expandable sheath towards the implantation site, and the expandable sheath can locally expand to accommodate the medical device as the device passes through. The radial force exerted by the medical device can be sufficient to locally expand the sheath to an expanded diameter (e.g., the expanded configuration) just in the area where the medical device is currently located. Once the medical device passes a particular location of the sheath, the sheath can at least partially contract to the smaller diameter of its original configuration. The expandable sheath can thus be expanded without the use of inflatable balloons or other dilators. Once the medical device is implanted, the sheath and any sutures holding in place can be removed. In some exemplary aspects, the sheath is removed without rotating it.

In view of the many possible aspects to which the principles of the disclosed invention can be applied, it should be recognized that the illustrated aspects are only some examples of the invention and should not be taken as limiting the scope of the invention. Rather, the scope of the invention is defined by the following claims. We, therefore, claim as our invention all that comes within the scope and spirit of these claims.

### EXEMPLARY ASPECTS

EXAMPLE 1: A sheath for delivering a medical device, wherein the sheath has a proximal end and a distal end; wherein the sheath has a collapsed unexpanded state and is configured to expand to an expanded state under passage of the medical device and resiliently return to the collapsed unexpanded state after passage of the medical device, wherein the sheath comprises: a) an inner layer comprising: i) a reinforcing layer comprising a plurality of struts axially extending from the proximal end to the distal end and circumferentially arranged in a predetermined pattern; ii) a polymer layer disposed around the plurality of struts to form a plurality of encapsulated struts; and wherein the polymer layer further comprises a plurality of tether portions circumferentially connecting each of the plurality of struts to each other; wherein when the sheath is in the unexpanded state: the plurality of encapsulated struts are configured to collapse to a randomized or an aligned organization to form the sheath defining a cross-section having a first diameter; wherein the plurality of tether portions at least partially folded between two adjacent struts of the plurality of encapsulated struts; and wherein the sheath is in the expanded state: the plurality of encapsulated struts and the plurality of tether portions together configured to organize to form the sheath having a substantially circular cross-section having a second diameter; wherein the second diameter is larger than the first diameter; and b) an outer layer disposed over the inner layer of the sheath and configured to maintain a general shape of the sheath; wherein the sheath is configured to resist axial elongational of the sheath such that a length of the sheath remains substantially constant.

EXAMPLE 2: The sheath of any examples herein, particularly example 1, wherein each of the plurality of struts is radially spaced from each other at a predetermined length.

EXAMPLE 3: The sheath of any examples herein, particularly example 2, wherein the predetermined length is identical between each of the plurality of struts.

EXAMPLE 4: The sheath of any examples herein, particularly example 2, wherein the predetermined length varies between any of the plurality of struts.

EXAMPLE 5: The sheath of any examples herein, particularly example 3, wherein the predetermined length between the plurality of struts is the same along the length of the sheath.

EXAMPLE 6: The sheath of any examples herein, particularly example 3, wherein the sheath comprises at least one portion where an identical predetermined length between the plurality of struts is different from an identical predetermined length between the plurality of struts in at least one other portion of the sheath.

EXAMPLE 7: The sheath of any examples herein, particularly example 4, wherein a variation in the predetermined length between any of the plurality of struts is the same along the length of the sheath.

EXAMPLE 8: The sheath of any examples herein, particularly example 4, wherein a variation in the predetermined length between any of the plurality of struts is different along various portions of the sheath.

EXAMPLE 9: The sheath of any examples herein, particularly examples 2-8, wherein a length of each of the plurality of tether portions of the polymer layer defines the predetermined length.

EXAMPLE 10: The sheath of any examples herein, particularly examples 1-9, wherein at least a portion of the plurality of struts comprises struts that are not coupled to any adjacent struts other than through the circumferentially connecting tethering portions of the polymer layer encapsulating the plurality of struts.

EXAMPLE 11: The sheath of any examples herein, particularly examples 1-9, wherein at least a portion of the plurality of struts comprises struts that are at least partially coupled to at least a portion of an adjacent strut by at least one bridging member.

EXAMPLE 12: The sheath of any examples herein, particularly examples 1-11, wherein a distal end of the reinforcing layer is different from a proximal end of the reinforcing layer.

EXAMPLE 13: The sheath of any examples herein, particularly example 12, wherein the plurality of struts at the proximal end of the reinforcing layer are coupled to each other circumferentially by at least one radially extending terminal strut.

EXAMPLE 14: The sheath of any examples herein, particularly example 13, wherein the sheath comprises a plurality of extension struts extending out and perpendicularly to the terminal strut and configured to secure the reinforcing layer to a hub.

EXAMPLE 15: The sheath of any examples herein, particularly example 14, wherein the plurality of extension struts are flared out radially.

EXAMPLE 16: The sheath of any examples herein, particularly example 14 or 15, wherein the plurality of extension struts have a T-bar shape.

EXAMPLE 17: The sheath of any examples herein, particularly examples 14 or 15, wherein the plurality of extension struts have an island shape.

EXAMPLE 18: The sheath of any examples herein, particularly examples 12-17, wherein the plurality of struts at the distal end of the reinforcing layer are coupled.

EXAMPLE 19: The sheath of any examples herein, particularly example 18, wherein the distal end of the reinforcing layer is tapered in relative to other portions of the reinforcing layer.

EXAMPLE 20: The sheath of any examples herein, particularly examples 1-19, wherein the plurality of struts are flat.

EXAMPLE 21: The sheath of any examples herein, particularly examples 1-19, wherein the plurality of struts are round.

EXAMPLE 22: The sheath of any examples herein, particularly examples 1-19, wherein the plurality of struts have an irregular cross-section.

EXAMPLE 23: The sheath of any examples herein, particularly examples 20-22, wherein the reinforcing layer comprises a plurality of individual wires forming the plurality of struts.

EXAMPLE 24: The sheath of any examples herein, particularly examples 20-22, wherein the reinforcing layer is an etched sheet forming the plurality of struts.

EXAMPLE 25: The sheath of any examples herein, particularly examples 20-22, wherein the reinforcing layer is a laser-cut sheet forming the plurality of struts.

EXAMPLE 26: The sheath of any examples herein, particularly examples 20-22, wherein the reinforcing layer is a laser-cut tube forming the plurality of struts.

EXAMPLE 27: The sheath of any examples herein, particularly example 26, wherein the tube is hypotube.

EXAMPLE 28: The sheath of any examples herein, particularly example 21, wherein the plurality of struts have a diameter from about 0.001″ to about 0.020″.

EXAMPLE 29: The sheath of any examples herein, particularly example 28, wherein each of the plurality of struts has the same diameter.

EXAMPLE 30: The sheath of any examples herein, particularly example 28, wherein the plurality of struts comprise struts having a varying diameter.

EXAMPLE 31: The sheath of any examples herein, particularly example 28, wherein the plurality of struts have a uniform diameter along the length of the sheath.

EXAMPLE 32: The sheath of any examples herein, particularly example 28, wherein at least one strut of the plurality of struts has a diameter varying along the length of the sheath.

EXAMPLE 33: The sheath of any examples herein, particularly example 20, wherein the plurality of struts have a width from about 0.001″ to about 0.100″ and a thickness from about 0.001″ to about 0.040″.

EXAMPLE 34: The sheath of any examples herein, particularly example 33, wherein the width and/or the thickness of each strut of the plurality of struts are substantially similar.

EXAMPLE 35: The sheath of any examples herein, particularly example 33, wherein the width and/or the thickness of the plurality of struts are uniform along the length of the sheath.

EXAMPLE 36: The sheath of any examples herein, particularly example 33, wherein the plurality of struts comprise struts having a varying width and/or thickness.

EXAMPLE 37: The sheath of any examples herein, particularly example 33, wherein at least one strut of the plurality of struts has a width and/or thickness varying along the length of the sheath.

EXAMPLE 38: The sheath of any examples herein, particularly examples 1-37, wherein the reinforcing layer comprises a metal or a polymer.

EXAMPLE 39: The sheath of any examples herein, particularly example 38, wherein the polymer comprises PEEK, nylon, or a combination thereof.

EXAMPLE 40: The sheath of any examples herein, particularly example 38 or 39, wherein the polymer exhibits a modulus between about 1 GPa to about 10 GPa.

EXAMPLE 41: The sheath of any examples herein, particularly examples 38-40, wherein the polymer has a Shore hardness from about 10 A to about 90 A or from about 10 D to about 90 D.

EXAMPLE 42: The sheath of any examples herein, particularly example 38, wherein the metal is titanium metal, nitinol, cobalt-chromium alloy, stainless steel, or any combinations or alloys thereof.

EXAMPLE 43: The sheath of any examples herein, particularly example 42, wherein the metal exhibits a modulus of about 20 GPa to about 250 GPa.

EXAMPLE 44: The sheath of any examples herein, particularly examples 1-43, wherein the plurality of struts comprise four or more struts.

EXAMPLE 45: The sheath of any examples herein, particularly examples 1-44, wherein the sheath is configured to receive an introducer configured to reinforce a radial orientation of the plurality of encapsulated struts upon introducing the sheath into a body.

EXAMPLE 46: The sheath of any examples herein, particularly example 45, wherein the introducer comprises at least a partially grooved surface configured to match the radial orientation of the plurality of encapsulated struts.

EXAMPLE 47: The sheath of any examples herein, particularly examples 1-46, wherein the polymer layer is lubricious.

EXAMPLE 48: The sheath of any examples herein, particularly examples 1-47, wherein the polymer layer is substantially not stretchable.

EXAMPLE 49: The sheath of any examples herein, particularly examples 1-48, wherein the polymer layer comprises e-PTFE.

EXAMPLE 50: The sheath of any examples herein, particularly example 49, wherein the polymer layer is sintered with the reinforcing layer.

EXAMPLE 51: The sheath of any examples herein, particularly examples 1-50, wherein a combined thickness of the polymer layer and the reinforcing layer is from about 0.003″ to about 0.040″.

EXAMPLE 52: The sheath of any examples herein, particularly examples 1-51, wherein a thickness of each of the plurality of tether portions is less than about 0.001″ to about 0.030″.

EXAMPLE 53: The sheath of any examples herein, particularly examples 1-52, wherein the outer layer comprises an elastomeric material.

EXAMPLE 54: The sheath of any examples herein, particularly examples 1-53, wherein the outer layer comprises a compound comprising a heat shrinking material, a polyether block amide, a polyurethane, silicone, polyisoprene, or any combinations thereof present up to 100 wt % based on a total weight of the compound.

EXAMPLE 55: The sheath of any examples herein, particularly example 54, wherein the outer layer comprises PEBAX®.

EXAMPLE 56: The sheath of any examples herein, particularly examples 1-55, wherein the outer layer has a thickness from about 0.001″ to about 0.010″.

EXAMPLE 57: The sheath of any examples herein, particularly examples 1-56, wherein the outer layer has a Shore hardness of from about 25 Durometer to about 90 Durometer.

EXAMPLE 58: The sheath of any examples herein, particularly examples 54-57, wherein the compound further comprises an inorganic filler present in an amount of less than about 60 wt % based on the total weight of the compound.

EXAMPLE 59: The sheath of any examples herein, particularly examples 54-58, wherein the compound further comprises a solid lubricant present up to about 20 wt % based on the total weight of the compound.

EXAMPLE 60: The sheath of any examples herein, particularly examples 1-59, wherein the outer layer comprises one or more sublayers.

EXAMPLE 61: The sheath of any examples herein, particularly example 60, wherein each of the sublayers are the same or different.

EXAMPLE 62: The sheath of any examples herein, particularly examples 1-61, wherein the outer layer is configured to apply an inward radial force on the sheath, biasing the sheath toward the unexpanded state.

EXAMPLE 63: The sheath of any examples herein, particularly examples 1-62, wherein a tie layer is disposed between the inner layer and the outer layer of the sheath.

EXAMPLE 64: The sheath of any examples herein, particularly examples 1-63, wherein the sheath is substantially kink-resistant.

EXAMPLE 65: The sheath of any examples herein, particularly examples 1-64, wherein the sheath is configured to expand due to small movements of the plurality of struts and the plurality of tether portions and thereby reduce a frictional force upon passage of the medical device.

EXAMPLE 66: A method of making a sheath for delivering a medical device comprising: a) providing an inner layer comprising: i) a reinforcing layer comprising a plurality of struts axially extending from the proximal end to the distal end and circumferentially arranged in a predetermined pattern; and ii) a polymer layer disposed around the plurality of struts to form a plurality of encapsulated struts; and wherein the polymer layer further comprises a plurality of tether portions circumferentially connecting each of the plurality of struts to each other; b) disposing an outer layer around the inner layer such that the outer layer is configured to maintain a general shape of the sheath; such that when the formed sheath is in the unexpanded state: the plurality of encapsulated struts are configured to collapse to a randomized or an aligned organization to form a sheath defining a cross-section having a first diameter; wherein the plurality of tether portions at least partially folded between two adjacent struts of the plurality of encapsulated struts; and when the sheath is in the expanded state: the plurality of encapsulated struts and the plurality of tether portions together configured to organize to form a sheath having a substantially circular cross-section having a second diameter; wherein the second diameter is larger than the first diameter; and wherein the formed sheath is configured to resist axial elongational of the sheath such that a length of the sheath remains substantially constant.

EXAMPLE 67: The method of any examples herein, particularly example 66, wherein each of the plurality of struts is radially spaced from each other at a predetermined length.

EXAMPLE 68: The method of any examples herein, particularly example 67, wherein the predetermined length is identical between each of the plurality of struts.

EXAMPLE 69: The method of any examples herein, particularly example 67, wherein the predetermined length varies between any of the plurality of struts.

EXAMPLE 70: The method of any examples herein, particularly example 68, wherein the predetermined length between the plurality of struts is the same along the length of the sheath.

EXAMPLE 71: The method of any examples herein, particularly example 68, wherein the sheath comprises at least one portion where the identical predetermined length between the plurality of struts is different from the identical predetermined length between the plurality of struts in at least one other portion of the sheath.

EXAMPLE 72: The method of any examples herein, particularly example 69, wherein a variation in the predetermined length between any of the plurality of struts is the same along the length of the sheath.

EXAMPLE 73: The method of any examples herein, particularly example 69, wherein a variation in the predetermined length between any of the plurality of struts is different along various portions of the sheath.

EXAMPLE 74: The method of any examples herein, particularly examples 67-73, wherein a length of each of the plurality of tether portions of the polymer layer defines the predetermined length.

EXAMPLE 75: The method of any examples herein, particularly examples 66-74, wherein at least a portion of the plurality of struts comprises struts that are not coupled to any adjacent struts other than through the circumferentially connecting tethering portions of the polymer layer encapsulating the plurality of struts.

EXAMPLE 76: The method of any examples herein, particularly examples 66-74, wherein at least a portion of the plurality of struts comprises struts that are at least partially coupled to at least a portion of an adjacent strut by at least one bridging member.

EXAMPLE 77: The method of any examples herein, particularly examples 66-76, wherein a distal end of the reinforcing layer is different from a proximal end of the reinforcing layer.

EXAMPLE 78: The method of any examples herein, particularly example 77, wherein the plurality of struts at the proximal end of the reinforcing layer are coupled to each other circumferentially by at least one radially extending terminal strut.

EXAMPLE 79: The method of any examples herein, particularly example 78, wherein the sheath comprises a plurality of extension struts extending out and perpendicularly to the terminal strut and configured to secure the reinforcing layer to a hub.

EXAMPLE 80: The method of any examples herein, particularly example 79, wherein the plurality of extension struts are flared out radially.

EXAMPLE 81: The method of any examples herein, particularly examples 79 or 80, wherein the plurality of extension struts have a T-bar shape.

EXAMPLE 82: The method of any examples herein, particularly examples 79 or 80, wherein the plurality of extension struts have an island shape.

EXAMPLE 83: The method of any examples herein, particularly examples 77-82, wherein the plurality of struts at the distal end of the reinforcing layer are coupled.

EXAMPLE 84: The method of any examples herein, particularly example 83, wherein the distal end of the reinforcing layer is tapered in relative to other portions of the reinforcing layer.

EXAMPLE 5 The method of any examples herein, particularly examples 66-84, wherein the plurality of struts are flat.

EXAMPLE 86: The method of any examples herein, particularly examples 66-84, wherein the plurality of struts are round.

EXAMPLE 87: The method of any examples herein, particularly examples 66-84, wherein the plurality of struts have an irregular cross-section.

EXAMPLE 88: The method of any examples herein, particularly examples 85-87, wherein the reinforcing layer comprises a plurality of individual wires forming the plurality of struts.

EXAMPLE 89: The method of any examples herein, particularly examples 85-87, wherein the reinforcing layer is an etched sheet forming the plurality of struts.

EXAMPLE 90: The method of any examples herein, particularly examples 85-87, wherein the reinforcing layer is a laser-cut sheet forming the plurality of struts.

EXAMPLE 91: The method of any examples herein, particularly examples 85-87, wherein the reinforcing layer is a laser-cut tube forming the plurality of struts.

EXAMPLE 92: The method of any examples herein, particularly example 91, wherein the tube is hypotube.

EXAMPLE 93: The method of any examples herein, particularly example 86, wherein the plurality of struts have a diameter from about 0.001″ to about 0.020″.

EXAMPLE 94: The method of any examples herein, particularly example 93, wherein each of the plurality of struts has the same diameter.

EXAMPLE 95: The method of any examples herein, particularly example 93, wherein the plurality of struts comprise struts having a varying diameter.

EXAMPLE 96: The method of any examples herein, particularly example 93, wherein the plurality of struts have a uniform diameter along the length of the sheath.

EXAMPLE 97: The method of any examples herein, particularly example 93, wherein at least one strut of the plurality of struts has a diameter varying along the length of the sheath.

EXAMPLE 98: The method of any examples herein, particularly example 85, wherein the plurality of struts have a width from about 0.001″ to about 0.100″ and a thickness from about 0.001″ to about 0.040″.

EXAMPLE 99: The method of any examples herein, particularly example 98, wherein the width and/or the thickness of each strut of the plurality of struts are substantially similar.

EXAMPLE 100: The method of any examples herein, particularly example 98, wherein the width and/or the thickness of the plurality of struts are uniform along the length of the sheath.

EXAMPLE 101: The method of any examples herein, particularly example 98, wherein the plurality of struts comprise struts having a varying width and/or thickness.

EXAMPLE 102: The method of any examples herein, particularly example 98, wherein at least one strut of the plurality of struts has a width and/or thickness varying along the length of the sheath.

EXAMPLE 103: The method of any examples herein, particularly examples 66-102, wherein the reinforcing layer comprises a metal or a polymer.

EXAMPLE 104: The method of any examples herein, particularly example 103, wherein the polymer comprises PEEK, nylon, or a combination thereof.

EXAMPLE 105: The method of any examples herein, particularly example 103 or 104, wherein the polymer exhibits a modulus from about 1 GPa to about 10 GPa.

EXAMPLE 106: The method of any examples herein, particularly examples 103-105, wherein the polymer is a polymer has a Shore hardness of from about 10 A to about 90 A or from about 10 D to about 90 D.

EXAMPLE 107: The method of any examples herein, particularly example 103, wherein the metal is titanium metal, nitinol, cobalt-chromium alloy, stainless steel, or any combinations or alloys thereof.

EXAMPLE 108: The method of any examples herein, particularly example 107, wherein the metal exhibits a modulus from about 20 GPa to about 250 GPa.

EXAMPLE 109: The method of any examples herein, particularly examples 66-107, wherein the plurality of struts comprises four or more struts.

EXAMPLE 110: The method of any examples herein, particularly examples 66-109, wherein the sheath is configured to receive an introducer configured to reinforce a radial orientation of the plurality of encapsulated struts upon introducing the sheath into a body.

EXAMPLE 111: The method of any examples herein, particularly example 110, wherein the introducer comprises a grooved surface configured to match the radial orientation of the plurality of encapsulated struts.

EXAMPLE 112: The method of any examples herein, particularly examples 66-111, wherein the polymer layer is lubricious.

EXAMPLE 113: The method of any examples herein, particularly examples 66-112, wherein the polymer layer is substantially not stretchable.

EXAMPLE 114: The method of any examples herein, particularly examples 66-113, wherein the polymer layer comprises e-PTFE.

EXAMPLE 115: The method of any examples herein, particularly example 114, wherein the method comprises a step of sintering the polymer layer with the reinforcing layer.

EXAMPLE 116: The method of any examples herein, particularly examples 66-115, wherein a combined thickness of the polymer layer and the reinforcing layer is from about 0.003″ to about 0.040″.

EXAMPLE 117: The method of any examples herein, particularly examples 66-116, wherein a thickness of each of the plurality of tether portions is less than about 0.001″ to about 0.030″.

EXAMPLE 118: The method of any examples herein, particularly examples 66-117, wherein the outer layer comprises an elastomeric material.

EXAMPLE 119: The method of any examples herein, particularly examples 66-118, wherein the outer layer comprises a compound comprising a heat shrinking material, a polyether block amide, a polyurethane, silicone, polyisoprene, or any combinations thereof present up to 100 wt % based on a total weight of the compound.

EXAMPLE 120: The method of any examples herein, particularly example 119, wherein the outer layer comprises PEBAX®.

EXAMPLE 121: The method of any examples herein, particularly examples 66-120, wherein the outer layer has a thickness from about 3 taus to about 10 taus.

EXAMPLE 122: The method of any examples herein, particularly examples 66-121, wherein the outer layer has a Shore hardness of from about 25 Durometer to about 90 Durometer.

EXAMPLE 123: The method of any examples herein, particularly examples 119-122, wherein the compound further comprises an inorganic filler present in an amount of less than about 60 wt % based on the total weight of the compound.

EXAMPLE 124: The method of any examples herein, particularly examples 119-123, wherein the compound further comprises a solid lubricant present up to about 20 wt % based on the total weight of the compound.

EXAMPLE 125: The method of any examples herein, particularly examples 66-124, wherein the outer layer comprises one or more sublayers.

EXAMPLE 126: The method of any examples herein, particularly example 125, wherein each of the sublayers are the same or different.

EXAMPLE 127: The method of any examples herein, particularly examples 66-126, wherein the outer layer is configured to apply an inward radial force on the sheath, biasing the sheath toward the unexpanded state.

EXAMPLE 128: The method of any examples herein, particularly examples 66-127, comprising a step of disposing a tie layer between the inner layer and the outer layer of the sheath.

EXAMPLE 129: The method of any examples herein, particularly examples 66-128, wherein the sheath is substantially kink-resistant.

EXAMPLE 130: The method of any examples herein, particularly examples 64-129, wherein the sheath is configured to expand due to small movements of the plurality of struts and the plurality of tether portions and thereby reduce a frictional force upon passage of the medical device.

EXAMPLE 131: A method of delivering a medical device through a sheath, the method comprising: a) introducing the medical device into a proximal end of a collapsed sheath, wherein the sheath comprises an inner layer and an outer layer, wherein the inner layer comprises: i) a reinforcing layer comprising a plurality of struts axially extending from the proximal end to the distal end and circumferentially arranged in a predetermined pattern; and ii) a polymer layer disposed around the plurality of struts to form a plurality of encapsulated struts; and wherein the polymer layer further comprises a plurality of tether portions circumferentially connecting each of the plurality of struts to each other; wherein in the collapsed sheath, the plurality of encapsulated struts are in a randomized or an aligned organization forming a sheath defining a cross-section having a first diameter, and wherein the plurality of tether portions at least partially folded between two adjacent struts of the plurality of encapsulated struts; b) advancing the medical device through the sheath such that the medical device exerts a radially outward force on the inner layer, thereby organizing and radially orienting the plurality of encapsulated struts and the plurality of tether portions together forming a sheath having a substantially circular cross-section having a second diameter; wherein the second diameter is larger than the first diameter; and c) locally contracting the expanded sheath back to an unexpanded configuration by radially compressing the expanded portion with a radially inward bias of an outer layer that extends around the inner layer.

EXAMPLE 132: The method of any examples herein, particularly example 131, wherein prior to introducing the medical device, an introducer is inserted into the sheath to organize the plurality of encapsulated struts and the plurality of tether portions to form the substantially circular cross-section having a second diameter.

EXAMPLE 133: The method of any examples herein, particularly example 131 or 132, wherein the medical device is a prosthetic heart valve mounted in a radially crimped state on a delivery apparatus, and the act of advancing the medical device through the sheath comprises advancing the delivery apparatus and the prosthetic heart valve into the vasculature of a patient.

1. A sheath for delivering a medical device including:
a proximal end;
a distal end;
an inner layer comprising:
a reinforcing layer comprising a plurality of struts axially extending from the proximal end to the distal end and circumferentially arranged in a predetermined pattern;
a polymer layer disposed around the plurality of struts to form a plurality of encapsulated struts; and wherein the polymer layer further comprises a plurality of tether portions circumferentially connecting each of the plurality of struts to each other; and
an outer layer disposed over the inner layer of the sheath and configured to maintain a shape of the sheath;
wherein the sheath has a collapsed unexpanded state and is configured to expand to an expanded state under passage of a medical device and resiliently return to the collapsed unexpanded state after passage of a medical device;
wherein when the sheath is in the unexpanded state:
the plurality of encapsulated struts are configured to collapse to a randomized or an aligned organization to form the sheath defining a cross-section having a first diameter; and
wherein the plurality of tether portions are at least partially folded between two adjacent struts of the plurality of encapsulated struts;
wherein the sheath is in the expanded state the plurality of encapsulated struts and the plurality of tether portions together configured to organize to form the sheath having a substantially circular cross-section having a second diameter;
wherein the second diameter is larger than the first diameter;
wherein the sheath is configured to resist axial elongational of the sheath such that a length of the sheath remains substantially constant.

2. The sheath of claim 1, wherein each of the plurality of struts is radially spaced from each other at a predetermined length, where predetermined length is identical between each of the plurality of struts.

3. The sheath of claim 1, wherein each of the plurality of struts is radially spaced from each other at a predetermined length, wherein the sheath comprises a first portion where the predetermined length between the plurality of struts is a first predetermined length, and a second portion where the predetermined length between the plurality of struts is a second predetermined length, the first predetermined length is different from the second predetermined length.

4. The sheath of claim 1, wherein each of the plurality of struts is radially spaced from each other at a predetermined length, where the predetermined length varies between any of the plurality of struts,
wherein a variation in the predetermined length between any of the plurality of struts is different along various portions of the sheath.

5. The sheath of claim 1, wherein at least a portion of the plurality of struts comprises struts that are at least partially coupled to at least a portion of an adjacent strut by at least one bridging member.

6. The sheath of claim 1, wherein a distal end of the reinforcing layer is different from a proximal end of the reinforcing layer, such that the plurality of struts at the proximal end of the reinforcing layer are coupled to each other circumferentially by at least one radially extending terminal strut.

7. The sheath of claim 6, wherein the sheath comprises a plurality of extension struts extending out and perpendicularly to the at least one radially extending terminal strut and configured to secure the reinforcing layer to a hub.

8. The sheath of claim 7, wherein the plurality of extension struts are flared out radially.

9. The sheath of claim 6, wherein the plurality of struts at the distal end of the reinforcing layer are coupled.

10. The sheath of claim 9, wherein the distal end of the reinforcing layer is tapered in relative to other portions of the reinforcing layer.

11. The sheath of claim 1, wherein the reinforcing layer comprises: at least one of a plurality of individual wires forming the plurality of struts, an etched sheet forming the plurality of struts, a laser-cut sheet forming the plurality of struts, or a laser-cut tube forming the plurality of struts.

12. The sheath of claim 1, wherein the sheath is configured to receive an introducer, the introducer reinforcing a radial orientation of the plurality of encapsulated struts upon introducing the sheath into a body.

13. The sheath of claim 12, wherein the introducer comprises at least a partially grooved surface configured to match the radial orientation of the plurality of encapsulated struts.

14. The sheath of claim 1, wherein the polymer layer is sintered with the reinforcing layer.

15. The sheath of claim 1, wherein the outer layer is configured to apply an inward radial force on the sheath, biasing the sheath toward the unexpanded state.

16. The sheath of claim 1, wherein a tie layer is disposed between the inner layer and the outer layer of the sheath.

17. The sheath of claim 1, wherein the sheath is configured to expand due to small movements of the plurality of struts and the plurality of tether portions and thereby reduce a frictional force upon passage of a medical device.

18. A method of delivering a medical device through a sheath, the method comprising:
introducing the medical device into a proximal end of a sheath in a collapsed configuration, wherein the sheath comprises an inner layer and an outer layer,
wherein the inner layer comprises:
a reinforcing layer comprising a plurality of struts axially extending from the proximal end to a distal end of the sheath, the plurality of struts circumferentially arranged in a predetermined pattern; and
a polymer layer disposed around the plurality of struts to form a plurality of encapsulated struts; and wherein the polymer layer further comprises a plurality of tether portions circumferentially connecting each of the plurality of struts to each other;
wherein the sheath in the collapsed configuration, the plurality of encapsulated struts are organized in at least one of a randomized or an aligned organization and define a cross-section of the sheath having a first diameter, and wherein the plurality of tether portions at least partially folded between two adjacent struts of the plurality of encapsulated struts;
advancing the medical device through the sheath such that the medical device exerts a radially outward force on the inner layer, thereby organizing and radially orienting the plurality of encapsulated struts and the plurality of tether portions together forming a substantially circular cross-section having a second diameter; wherein the second diameter is larger than the first diameter; and
locally contracting the expanded sheath back towards the unexpanded configuration by radially compressing an expanded portion of the sheath with a radially inward bias of an outer layer that extends around the inner layer.

19. The method of claim 18, wherein prior to introducing the medical device, an introducer is inserted into the sheath to organize the plurality of encapsulated struts and the plurality of tether portions to form the substantially circular cross-section having a second diameter.

20. The method of claim 18, wherein the medical device is a prosthetic heart valve mounted in a radially crimped state on a delivery apparatus, and advancing the medical device through the sheath comprises advancing the delivery apparatus and the prosthetic heart valve into a vasculature of a patient.
